(19) United States
(12) Patent Application Publication
(10) Pub. No.: US 2024/0327442 A1
(43) Pub. Date: Oct. 3, 2024

(54) CONTROLLING METAL-ORGANIC FRAMEWORK MORPHOLOGY THROUGH COORDINATIVE MIMICRY

(71) Applicants: THE REGENTS OF THE UNIVERSITY OF CALIFORNIA, Oakland, CA (US); EXXONMOBIL TECHNOLOGY AND ENGINEERING COMPANY, Annandale, NJ (US)

(72) Inventors: Megan N. Jackson, Berkeley, CA (US); Jeffrey R. Long, Oakland, CA (US); Joseph M. Falkowski, Hampton, NJ (US)

(21) Appl. No.: 18/294,067

(22) PCT Filed: Jul. 21, 2022
(86) PCT No.: PCT/US2022/037901

§ 371 Date: Jan. 31, 2024

Related U.S. Application Data
(60) Provisional application No. 63/228,503, filed on Aug. 2, 2021.

(51) Int. Cl. C07F 15/06

(52) U.S. Cl. CPC C07F 15/065

(57) ABSTRACT

Methods of synthesizing crystalline metal-organic frameworks (MOFs) having polytopic organic linkers and, cations, where each linker is connected, to two or more cations, are provided. In the disclosed methods, the linkers are reacted with one or more compounds of formula MₙXₘ, where each M is independently cationic Be, Mg, Ca, Ti, V, Cr, Mn, Fe, Co, Ni, Cu, Zn, Zr, Nb, Mo, Ru, Rh, Pd, Cd, or Hf, X is anionic, n and m are integers. The reaction is performed in the presence of a salicylate, salicylamide, 1,3-phthalate, or hydroxybenzoate based modulator.

### CROSS REFERENCE TO RELATED APPLICATIONS

The present disclosure claims priority to U.S. Provisional Patent Application No. 63/228,503, filed on Aug. 2, 2021, which is hereby incorporated by reference in its entirety.

### FIELD OF THE PRESENT DISCLOSURE

The present application relates to the synthesis of metal-organic framework (MOF) crystals under conditions that provide advantageous crystalline morphology thereby improving the bulk performance of such crystallized MOFs.

### BACKGROUND

Porous materials have applicability as adsorbents and catalysts in a wide range of technologies such as chemical separations, energy storage, catalysis, drug delivery, and sensing, among others. Potential industrial applications of a particular class of porous materials, metal-organic frameworks, include methane conversion, hydrocarbon separations and catalysis, noble gas separations, and carbon dioxide capture from flue gas. See, for example, Li et al., 2011, “Metal-Organic Frameworks for Separations,” Chem. Rev. 112, 869; Sumida et al., 2012, “Carbon Dioxide Capture in Metal-Organic Frameworks,” Chem. Rev. 112, 724; McDonald et al., 2015, “Cooperative Insertion of CO₂ in Diamine-Appended Metal-Organic Frameworks,” Nature 519, 303; Milner et al., 2018, “Overcoming double-step CO₂ adsorption and minimizing water co-adsorption in bulky diamine-appended variants of Mg₂(4,4′-dioxidobiphenyl-3,3′-dicarboxylate),” Chem. Sci. 9, 160; and Bachman et al., 2016, “Enhanced ethylene separation and plasticization resistance in polymer membranes incorporating metal-organic framework nanocrystals,” Nature Mater. 15, 845.

The processes involved in such chemical separations currently account for 10-15% of the world's energy usage. See. 2005, Oak Ridge National Laboratory. Materials for Separation Technologies: Energy and Emission Reduction Opportunities; and Humphrey and Keller, 1997, Separation Process Technology, McGraw-Hill. Separation performance, such as in packed-bed applications, can be highly dependent upon crystallite size and shape, which collectively control the surface area-to-volume ratio and mass transfer resistances of porous materials such as metal-organic frameworks. See Rousseau, 1987, “Handbook of Separation Process Technology,” John Wiley and Sons, pp. 669-671. Catalytic performance of heterogeneous catalysts, such as metal-organic frameworks, can derive from factors including material phase and mass transfer resistances, the latter of which can be a function of crystallite size and shape. See Fogler, 2016, Elements of Chemical Reaction Engineering, Fifth Ed., Prentice Hall.

Adsorbents, such as M₂(dobdc) (M=Mg, Mn, Fe, Co, Ni, Zn, Cd; dobdc⁴⁻=2,5-dioxido-1,4-benzenedicarboxylate, FIG. 20) and the associated expanded family of materials M₂(dobpdc) (M=Mg, Mn, Fe. Co, Ni, Zn; dobpdc=4,4′-dioxidobiphenyl-3,3′-dicarboxylate, FIG. 21) (McDonald et al., 2015, Nature 519, p. 303; Siegelman, 2017, J. Am. Chem. Soc., 139, p. 10526) are of interest as porous solid adsorbent materials for use in separations, catalytic applications, and gas storage. Such metal-organic frameworks are of interest due to the structure featuring coordinatively-unsaturated metal sites along the pore. See Rosi et al., 2005, J. Am. Chem. Soc. 127(5), 1504; Rowsell et al., 2006, Am. Chem. Soc. 128, p. 1304; Caskey et al., 2008, J. Am. Chem. Soc. 130, p. 10870; McDonald et al., 2012, J. Am. Chem. Soc. 134, p. 7056; and Long et al., International Publication No. WO2013059527 A1, entitled “Alkylamine functionalized metal-organic frameworks for composite gas separations,” dated Apr. 25, 2013.

One drawback with these materials is that conventional synthetic schemes typically result in crystalline metal-organic framework in which there is extended anisotropic growth, resulting in rod-like crystalline product. As illustrated in FIG. 1, metal-organic framework syntheses typically require a source of metal ions or clusters and a partially-to-fully deprotonated ligand. Generally, this is done at elevated temperature and in solution, with deprotonation occurring via formation of a base from solvent decomposition. The solvent is often a toxic and/or expensive solvent such as N,N-dimethylformamide.

Because macroscale crystalline characteristics of metal-organic frameworks, such as size and shape, have significant influence on adsorbent performance, such anisotropic crystal structures arising from conventional metal-organic framework schemes often preclude significant diffusion in the direction of the ab plane, for example in M₂(dobdc) and M₂(dobpdc) compounds. See, for instance, Colwell et al., “Buffered Coordination Modulation as a Means of Controlling Crystal Morphology and Molecular Diffusion in an Anisotropic Metal-Organic Framework,” J. Am. Chem. Soc. 2021, 143, 13, 5044-5052; and Forse et al., “Influence of Pore Size on Carbon Dioxide Diffusion in Two Isoreticular Metal-Organic Frameworks,” Chem. Mater. 2020, 32, 8, 3570-3576. In other cases, the direction of diffusion is dependent on the respective metal-organic frameworks. Because of this, the final metal-organic framework scheme of a respective crystal has a large impact on diffusion rates through the crystal.

As such, one goal in MOF synthesis is to establish the synthesis conditions that lead to crystalline metal-organic frameworks without decomposition of the organic linker. At the same time, the kinetics of crystallization should be appropriate to allow nucleation and growth of the desired phase to take place. These complex relationships make it difficult to determine synthetic reaction conditions for MOFs that will yield suitably sized and shaped MOF crystallites.

In previous work on MOF fundamentals, synthesis utilizing non-coordinating buffers has been used to independently control reaction pH during metal-organic framework synthesis, enabling direct interrogation of the role of the coordinating species on crystal growth. This work demonstrated the efficacy of using buffered reaction conditions in the synthesis of low-dispersity single-crystals of the framework Co₂(dobdc) in a pH 7-buffered solution using cobalt(II) carboxylate salts as the metal sources. The work found that the aspect ratio of Co₂(dobdc) crystals had an inverse correlation with the pKₐ of the carboxylate modulator used in the synthesis. See International Patent Publication Number WO2020/068996 entitled “Metal-Organic Framework Phase and Crystallite Shape Control.”

As illustrated in FIGS. 2-4, another strategy used in the literature to affect a specific size or shape of metal-organic framework crystallite is using additives with an established synthesis, referred to as coordination modulators. See Stock and Biswas, 2012, Chem. Rev. 112, 933; Hermes et al., 2007 J. Am. Chem. Soc. 129, 5324; Cho et al., 2008, J. Am. Chem. Soc. 130, 16943; Diring et al., 2010, Chem. Mater. 22, 4531; and Pachfule et al., 2016, Nature Chem. 8, 718. Most of these additives have the same or similar functional groups as the organic linker and are presumed to competitively bind during growth. However, all of these additives are also acids or bases, and can participate in pH equilibria during growth in addition to coordination equilibria. The only example of M₂(dobdc) control uses salicylic acid as a modulator but does not address its participation in acid/base equilibria. See Pachfule et al., 2016, Nature Chem. 8, 718. Other published syntheses of M₂(dobdc) or M₂(dobpdc) do not form monodisperse samples of crystallites, and they are of high aspect ratio or form polycrystalline masses. See Rosi et al., 2005, J. Am. Chem. Soc. 127(5), 1504, Rowsell and Yaghi, 2006, Am. Chem. Soc. 128, 1304; and Caskey et al., 2008, J. Am. Chem. Soc. 130, 10870. One limitation of substituted carboxylate modulators is that they only modulate crystal morphology along the c-axis of the crystal. Based on zero-length chromatography and NMR diffusivity studies, gas diffuses faster through the pores along the c-axis of M₂(dobdc) crystals than along the ab-planes (see, Forse et al., 2020, “influence of Pore Size on Carbon Dioxide Diffusion in Two Isoreticular Metal-Organic Frameworks,” Chem. Mater. 2020, 32 (8), 3570-3576), suggesting that shorter disk shapes would be ideal for fast gas diffusion, and long needles would be ideal for slow gas diffusion. In addition, FIG. 5 illustrates how the basic anion used governs MOF crystal morphology in accordance with the prior art. FIG. 6 illustrates the use of different basic anions coupled with pH control to synthesize MOF crystals in accordance with the prior art.

### SUMMARY

Accordingly, given the above background, what is needed in the art are improved metal-organic framework synthetic schemes that result in crystalline product with controlled crystallite dimensions. For instance, what is needed in the art is new synthetic methods for controlling the morphology of crystals of MOFs, such as crystals of M₂(dobdc), beyond the c-axis.

Disclosed herein are synthetic schemes for controlling the shape of crystals of metal-organic frameworks (MOFs) beyond just the c-axis through the judicious choice of modulators present in the synthesis of such metal-organic frameworks. The present disclosure makes use of modulators such as salicylates, salicylamides, 1,3-phthalates, and 3-hydroxybenzoates to yield a range of new MOF crystal morphologies. The present disclosure provides synthetic conditions that synthesize MOF crystals with needle, disk, or rice grain shapes. The needle and disk shapes in particular are of interest because they enable a new method with which to tune gas diffusion rates through MOF crystals.

One aspect of the present disclosure provides a method of synthesizing a crystalline metal-organic framework comprising a plurality of cations and a plurality of polytopic organic linkers. Each polytopic organic linker in the plurality of polytopic organic linkers is connected to two or more cations in the plurality of cations. In the method, the plurality of polytopic organic linkers is reacted with one or more compounds of formula MₙXₘ in a solution, where each M is independently cationic Be, Mg, Ca, Ti, V, Cr, Mn, Fe, Co, Ni, Cu, Zn, Zr, Nb, Mo, Ru, Rh, Pd, Cd, Hf, where X is a basic anion, where n is a positive integer, and where m is a positive integer. The reaction takes place in the presence of a modulator having the formula:

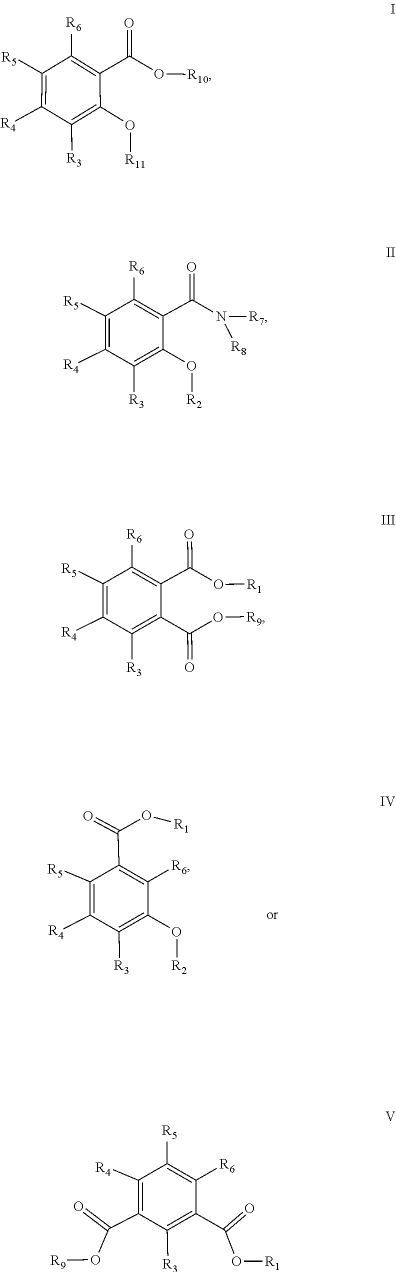

or a salt (e.g., sodium, potassium cesium) thereof or a mixture thereof, where R₁, R₂, R₇, R₈, R₉, R₁₀ and R₁₁ are each independently selected from hydrogen, halogen, substituted or unsubstituted alkyl, substituted or unsubstituted heteroalkyl, substituted or unsubstituted aryl, substituted or unsubstituted heteroaryl, or substituted or unsubstituted heterocycloalkyl, R₃, R₄, R₅, and R₆ are each independently selected from H, halogen, hydroxyl, methyl, or halogen substituted methyl, and where R₁₀ and R₁₁ are not each hydrogen.

These and other features and attributes of the disclosed synthetic schemes of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 illustrates how a metal-organic framework is formed from the combination of a metal cation and a ligand;

FIG. 2 illustrates how modulators can inhibit growth of metal-organic framework crystals;

FIG. 3 illustrates how modulators can control the size and shape of metal-organic framework crystals:

FIG. 4 illustrates mechanisms by which modulators can control the size and shape of metal-organic framework crystals;

FIG. 5 illustrates how the basic anion used governs MOF crystal morphology in accordance with the prior art:

FIG. 6 illustrates the differentiating roles of modulator and pH in metal-organic framework crystals;

FIG. 7 illustrates synthesis modulation to get larger isotropic nanocrystals of metal-organic frameworks in accordance with embodiments of the present disclosure:

FIGS. 8A, 8B, and 8C illustrate additional modulators for controlled growth of Co₂(dobdc) in accordance with embodiments of the present disclosure:

FIG. 9 illustrates conditions for synthesizing Co₂(dobdc) crystals in accordance with an embodiment of the present disclosure;

FIG. 10 illustrates, in clockwise fashion, Co₂(dobdc) crystals at 350× magnification grown in the presence of the modulators 3-hydroxybenzoate, acetate, salicylate, and phthalate in accordance with embodiments of the present disclosure;

FIG. 11 illustrates, in clockwise fashion, Co₂(dobdc) crystals at 3500× magnification grown in the presence of the modulators 3-hydroxybenzoate, acetate, salicylate, and phthalate in accordance with embodiments of the present disclosure;

FIG. 12 illustrates, in clockwise fashion, Co₂(dobdc) crystals at 5000× magnification grown in the presence of the modulators 3-hydroxybenzoate, acetate, salicylate, and phthalate in accordance with embodiments of the present disclosure;

FIG. 13 illustrates, in clockwise fashion, Co₂(dobdc) crystals at 8000× magnification grown in the presence of the modulators 3-hydroxybenzoate, acetate, salicylate, and phthalate in accordance with embodiments of the present disclosure;

FIG. 14 illustrates, in clockwise fashion, Co₂(dobdc) crystals at 10000× magnification grown in the presence of the modulators 3-hydroxybenzoate, acetate, salicylate, and phthalate in accordance with embodiments of the present disclosure;

FIG. 15 illustrates, in clockwise fashion, Co₂(dobdc) crystals at 12000× magnification grown in the presence of the modulators 3-hydroxybenzoate, acetate, salicylate, and phthalate in accordance with embodiments of the present disclosure:

FIG. 16 illustrates Co₂(dobdc) crystals grown in the presence of the modulator salicylate at 1504× magnification (top), and 3-hydroxybenzoate at 2000× magnification (bottom), in accordance with an embodiment of the present disclosure;

FIG. 17 illustrates consistently smaller crystals at 10 equivalents of 3-hydroxybenzoate than 1 equivalent, but crystal size does not monotonically decrease with modulator concentration, with the same trends observed for salicylamide, phthalate, and bromosalicylamide, in accordance with an embodiment of the present disclosure;

FIG. 18 illustrates how higher pH leads to lower aspect ratio crystals in accordance with an embodiment of the present disclosure;

FIG. 19 illustrates how modulator incorporation into the metal-organic framework is extremely low, in the case of trichloroacetate-modulated metal-organic framework crystallization, it is 83 ppm by elemental analysis (left panel) and it is not visible by energy dispersive X-ray spectroscopy, in accordance with an embodiment of the present disclosure:

FIG. 20 illustrates the structure of a metal-organic framework M₂(dobdc), consisting of divalent metal cations and the ligand H₄dobdc, in accordance with the prior art; and

FIG. 21 illustrates the structure of a metal-organic framework M₂(dobpdc), consisting of divalent metal cations and the ligand H₄dobpdc, in accordance with the prior art.

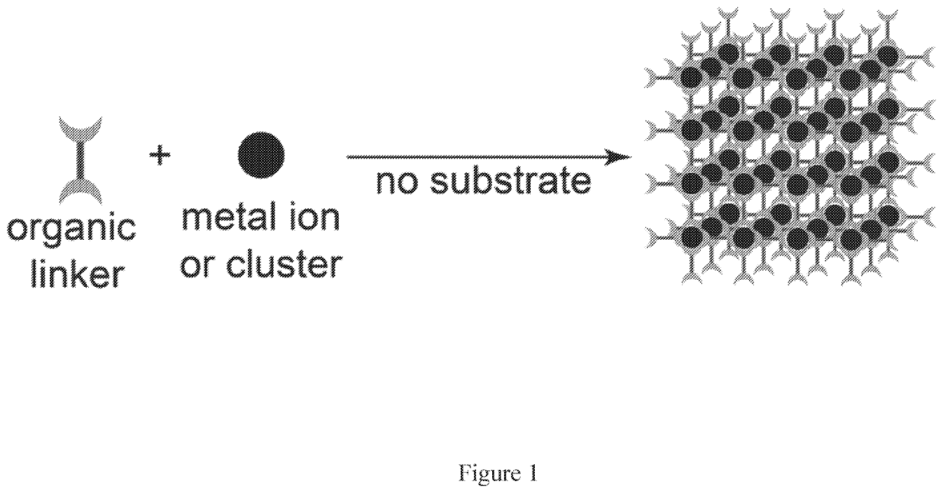

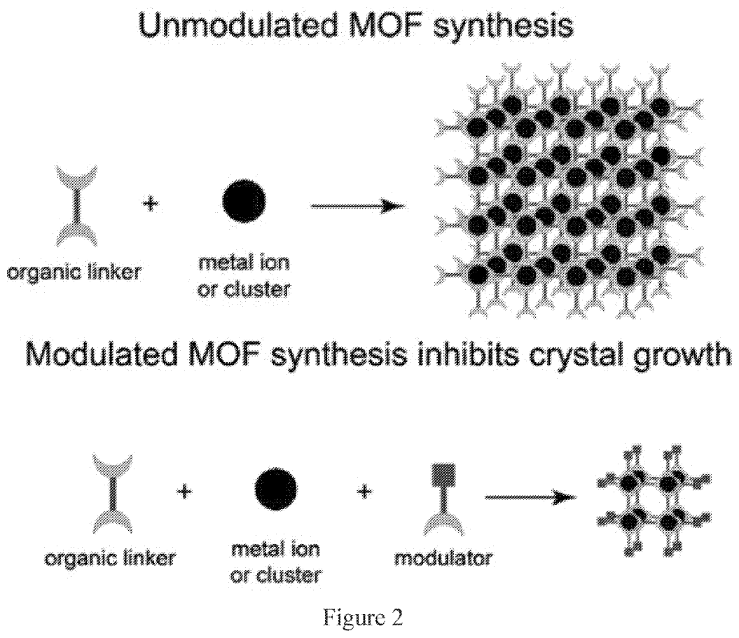

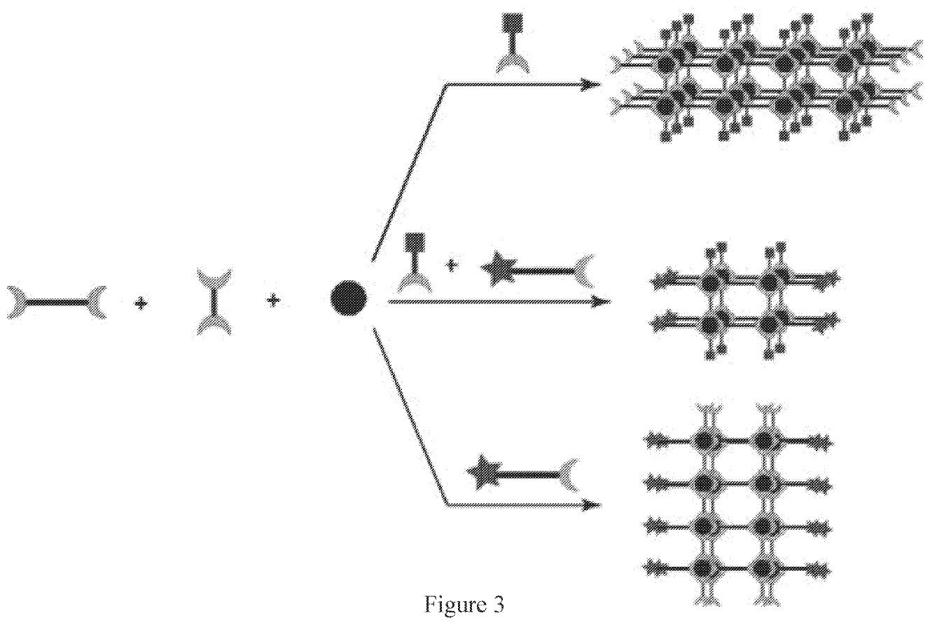

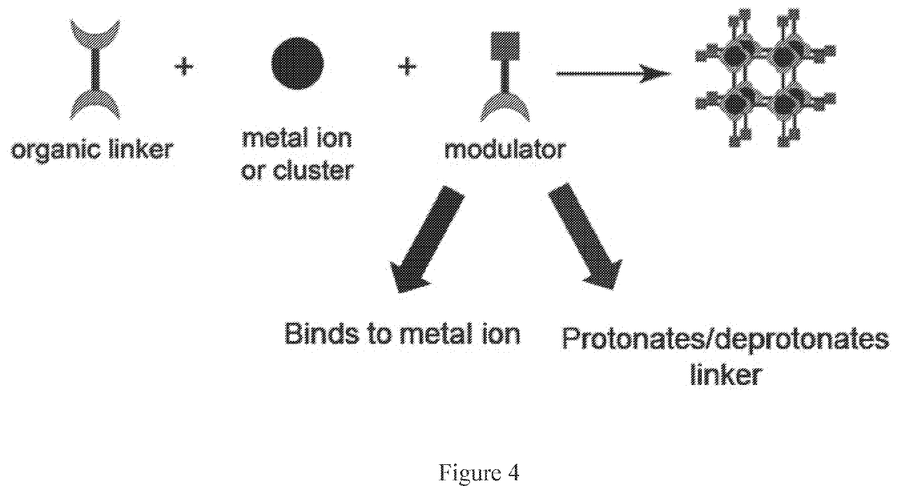

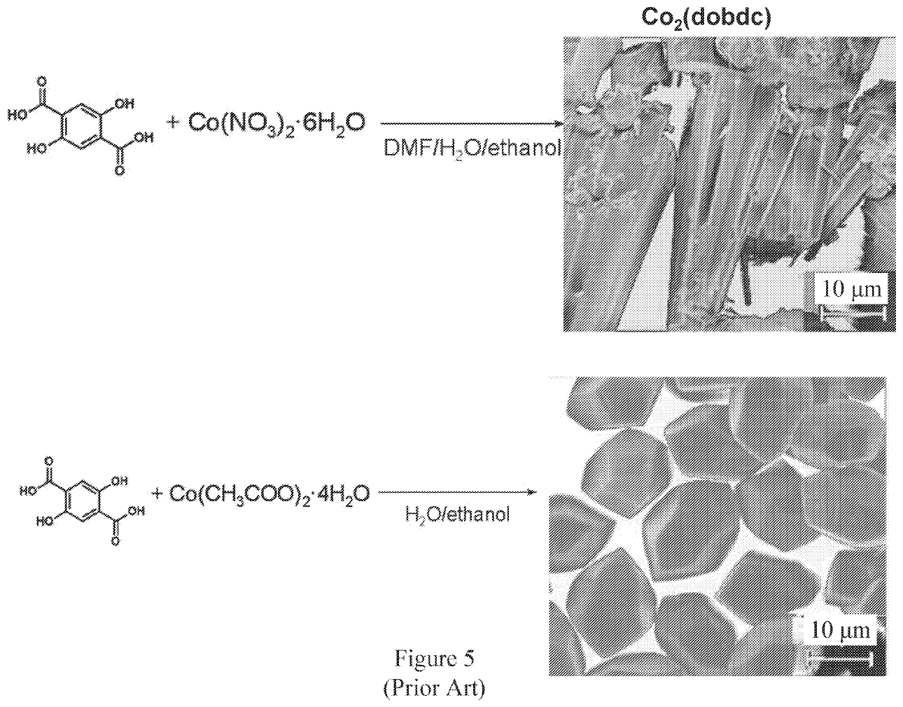

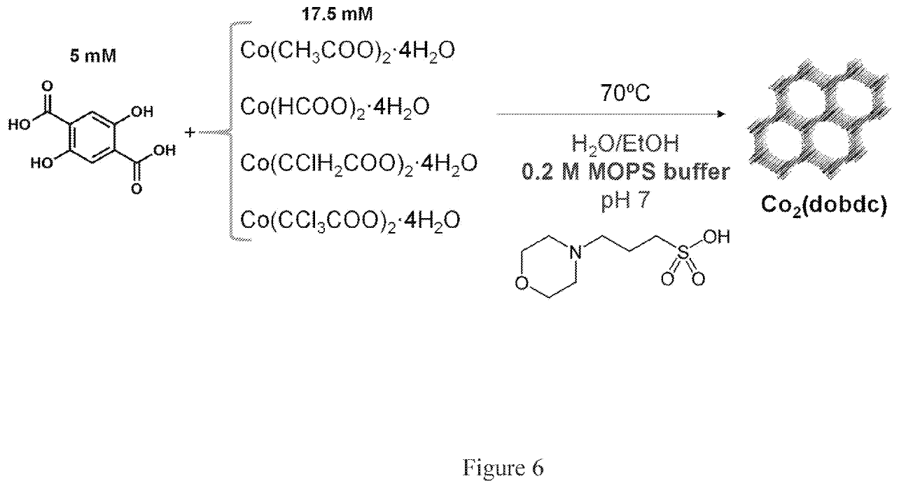

### DETAILED DESCRIPTION OF THE INVENTION

### I. Introduction

One drawback with conventional MOF crystal synthetic schemes is that they typically result in crystalline MOFs in which there is extended anisotropic growth, resulting in rod-like crystalline product. Because macroscale crystalline characteristics of MOFs, such as size and shape, have significant influence on adsorbent performance, such anisotropic crystal structures arising from conventional metal-organic framework schemes often preclude significant diffusion in the direction of the ab plane. Thus, most of the crystallite external surface for high aspect-ratio crystallites is expected to be inaccessible to gas diffusion. As such, MOF synthesis requires synthesis conditions that lead to crystalline MOFs without decomposition of the organic linker while, at the same time, promoting kinetics of crystallization that allow nucleation and growth of the desired phase to take place. These complex relationships make it difficult to determine synthetic reaction conditions for MOFs that will yield suitably sized and shaped MOF crystallites.

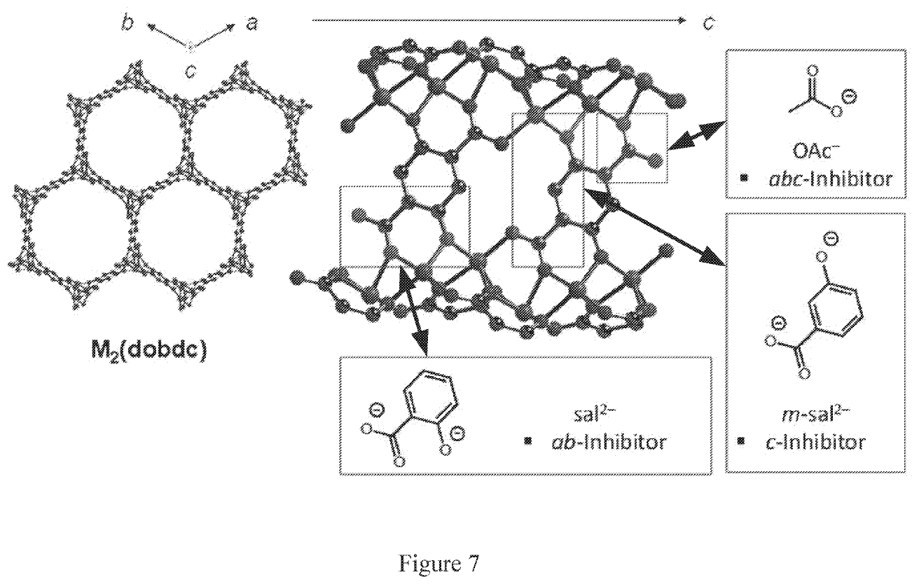

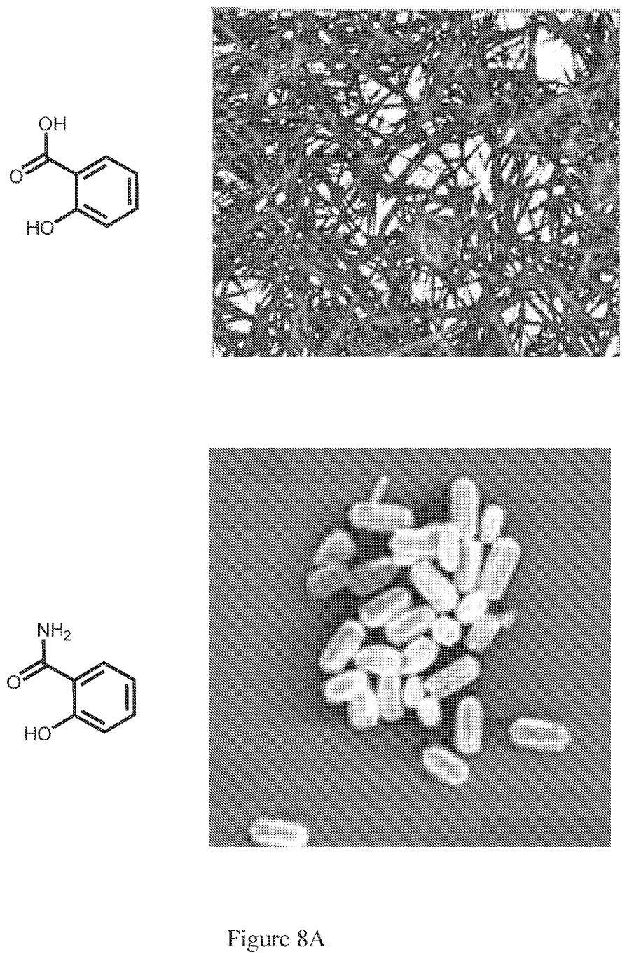

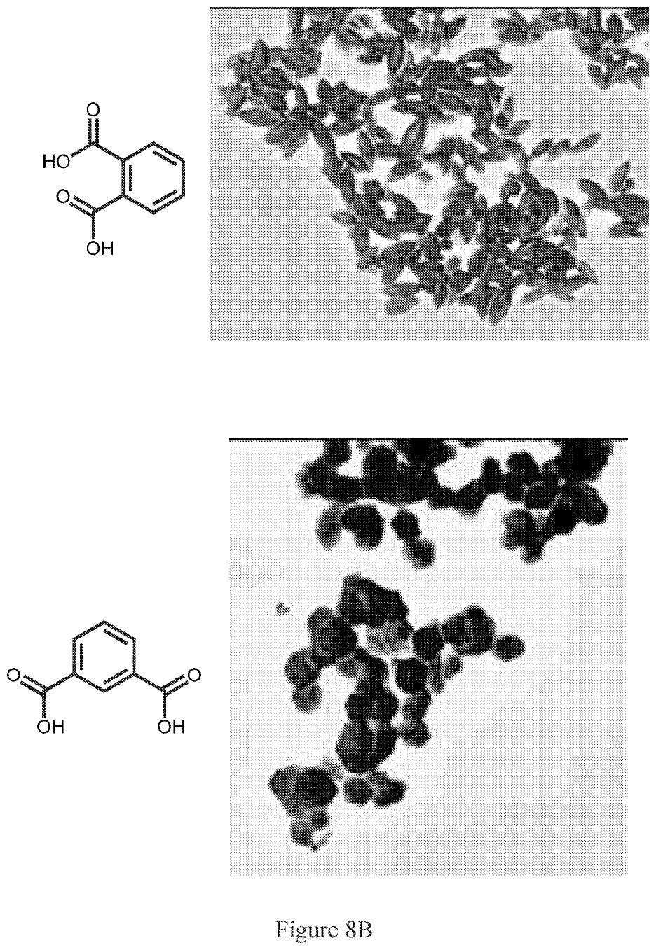

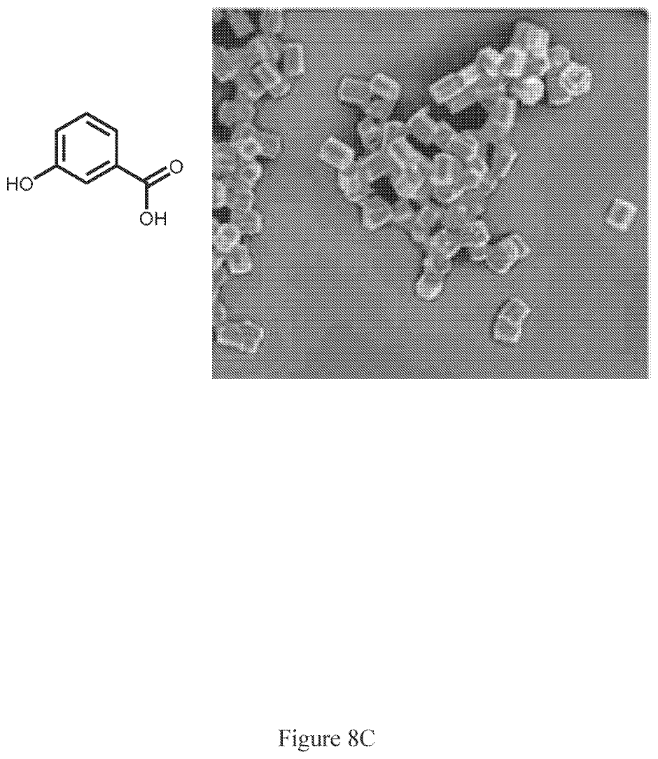

The present disclosure provides for the synthesis of crystalline metal-organic frameworks (MOFs). These MOFs comprise polytopic organic linkers and metal cations, in which each polytopic organic linker is connected to two or more of the metal cations. In the disclosed methods, the linkers are reacted with one or more compounds of formula MₙXₘ, where each M is independently cationic Be, Mg, Ca, Ti, V, Cr, Mn, Fe, Co, Ni, Cu, Zn, Zr, Nb, Mo, Ru, Rh, Pd, Cd, or Hf, X is a basic anion, and n is a positive integer (e.g., 1, 2, etc.) and m is a positive integer (e.g., 1, 2, etc.). In some embodiments, the reacting is in the presence of modulator such as a salicylate, salicylamide, 1,3-phthalate, 3-hydroxybenzoate or a salt (e.g., sodium, potassium cesium) thereof, or a mixture thereof. As illustrated in FIG. 7, each modulator type has an effect on a specific axis or plane, or on the overall size of MOF crystals. Moreover, as illustrated in FIGS. 8A, 8B, and 8C, this causes each modulator to have a distinct effect on MOF crystal morphology.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are defined as indicated below.

### II. Definitions

Where substituent groups are specified by their conventional chemical formulae, written from left to right, the structures optionally also encompass the chemically identical substituents, which would result from writing the structure from right to left, e.g., —CH₂O— is intended to also optionally recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, cyclohexylmethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl.” Exemplary alkyl groups include the monounsaturated C₉₋₁₀, oleoyl chain or the diunsaturated C_{9-10, 12-13} linoeyl chain.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The terms “aryloxy” and “heteroaryloxy” are used in their conventional sense, and refer to those aryl or heteroaryl groups attached to the remainder of the molecule via an oxygen atom.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N. Si and S. and wherein the nitrogen and sulfur atoms may optionally be oxidized and the nitrogen heteroatom may optionally be quaternized. The heteroatom(s) O, N, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH)—CH₃, —CH₂—S—CH₂—CH, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cvclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl substituent groups (or rings) that contain from one to four heteroatoms selected from N, O, S, Si and B, wherein the nitrogen and sulfur atoms are optionally oxidized, and the nitrogen atom(s) are optionally quaternized. An exemplary heteroaryl group is a six-membered azine, e.g., pyridinyl, diazinyl and triazinyl. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl. 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes aryl, heteroaryl and heteroarene rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl,” and “heteroaryl”) are meant to optionally include both substituted and unsubstituted forms of the indicated species. Exemplary substituents for these species are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R′″, OC(O)R′, —C(O)R′, —CO₂R′, —CONR′R″, —OC(O)NR′R″, —NR″C(O)R′, NR′ C(O)NR″R′″, —NR″C(O)2R′, —NR—C(NR′R″R′″)═NR′″, —NRC(NR′R″)═NR′″, —S(O)R′, —S(O)2R′, —S(O)2NR′R″, NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents”, which are components of exemplary “substituted alkyl” and “substituted heteroalkyl” moieties.

Similar to the substituents described for the alkyl radical, substituents for the aryl heteroaryl and heteroarene groups are generically referred to as “aryl group substituents.” The substituents are selected from, for example: groups attached to the heteroaryl or heteroarene nucleus through carbon or a heteroatom (e.g., P, N, O, S, Si, or B) including, without limitation, substituted or unsubstituted alkyl, substituted or unsubstituted aryl, substituted or unsubstituted heteroaryl, substituted or unsubstituted heterocycloalkyl, OR′, ═O, ═NR′, ═N—OR′, —NR′R″, —SR′, -halogen, —SiR′R″R′″, OC(O)R′, —C(O)R′, CO2R′, —CONR′R″, —OC(O)NR′R″, —NR″C(O)R′, NR′ C(O)NR″R″, —NR″C(O)2R′, NR—C(NR′R″R′″)═NR′″″, NR C(NR′R″)═NR′″, —S(O)R′, —S(O)2R′, —S(O)2NR′R″, NRSO2R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. Each of the above-named groups is attached to the heteroarene or heteroaryl nucleus directly or through a heteroatom (e.g., P, N, O, S, Si, or B); and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl, heteroarene or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)?NR′— or a single bond, and r is an integer of from 1 to 4. One of the single bonds of the new ring so formed may optionally be replaced with a double bond. Alternatively, two of the substituents on adjacent atoms of the aryl, heteroarene or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R″″ are preferably independently selected from hydrogen or substituted or unsubstituted (C₁-C₆) alkyl. These terms encompass groups considered exemplary “aryl group substituents”, which are components of exemplary “substituted aryl” “substituted heteroarene” and “substituted heteroaryl” moieties.

As used herein, the term “acyl” describes a substituent containing a carbonyl residue. C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non-aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si), boron (B) and phosphorous (P).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “salt(s)” includes salts of the compounds prepared by the neutralization of acids or bases, depending on the particular ligands or substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium salt, or a similar salt. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic, propionic, isobutyric, butyric, maleic, malic, malonic, benzoic, succinic, suberic, fumaric, lactic, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts. Hydrates of the salts are also included.

“—COOH” as this term is used is meant to optionally include —C(O)O⁻ and —C(O)O⁻X⁺, wherein X⁺ is a cationic counter-ion. Likewise, a substituent having the formula —N(R)(R) is meant to optionally include —N⁺H(R)(R) and —N⁺H(R)(R)Y⁻, where Y⁻ represents an anionic counter-ion. Exemplary polymers of the invention include a protonated carboxylic moiety (COOH). Exemplary polymers of the invention include a deprotonated carboxylic moiety (COO⁻). Various polymers of the invention include both a protonated carboxylic moiety and a deprotonated carboxylic moiety.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

Below are examples of specific embodiments of the present disclosure. The examples are offered for illustrative purposes only, and are not intended to limit the scope of the present invention in any way.

### III. Compositions

One aspect of the present disclosure provides a crystalline material. The crystalline material comprises a metal-organic framework comprising a plurality of metal ions and a plurality of polytopic organic linkers. Each polytopic organic linker in the plurality of polytopic organic linkers is connected to at least two metal ions in the plurality of metal ions. In some embodiments, the adsorption material further comprises a plurality of ligands. In some such embodiments, each respective ligand in the plurality of ligands is appended to a metal ion in the plurality of metal ions of the metal-organic framework. In some embodiments, the crystalline material is in the form of discrete crystals with needle, disk, or rice grain shape.

In some embodiments, the polytopic organic linker is 2,5-dioxido-1,4-benzenedicarboxylate (dobdc⁴⁻), 4,6-dioxido-1,3-benzenedicarboxylate (m-dobdc⁴⁻), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), or dioxidobiphenyl-4,4′-dicarboxylate (para-carboxylate-dobpdc⁴⁻ also referred to as pc-dobpdc⁴⁻).

In some embodiments, each polytopic organic linker in the plurality of polytopic organic linkers has the formula:

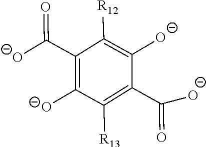

where R₁₂ and R₁₃ are each independently selected from H, halogen, hydroxyl, methyl, or halogen substituted methyl. In some embodiments, R₁₂ and R₁₃ are each hydrogen. In some embodiments, the polytopic organic linker is unprotonated as illustrated, partially protonated, or fully protonated in the solution.

In some embodiments, each polytopic organic linker in the plurality of polytopic organic linkers has the formula:

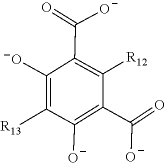

where R₁₂ and R₁₃ are each independently selected from H, halogen, hydroxyl, methyl, or halogen substituted methyl. In some embodiments, R₁₂ and R₁₃ are each hydrogen. In some embodiments, the polytopic organic linker is unprotonated as illustrated, partially protonated, or fully protonated in the solution.

In some embodiments, each polytopic organic linker in the plurality of polytopic organic linkers has the formula:

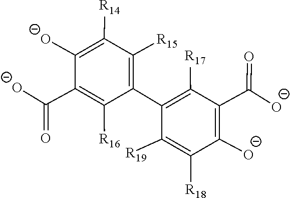

where R₁₄, R₁₅, R₁₆, R₁₇, R₁₈, and R₁₉ are each independently selected from H, halogen, hydroxyl, methyl, or halogen substituted methyl. In some embodiments, R₁₄, R₁₅, R₁₆, R₁₇, R₁₈, and R₁₉ are each hydrogen. In some embodiments, the polytopic organic linker is unprotonated as illustrated, partially protonated, or fully protonated in the solution.

In some embodiments, each polytopic organic linker in the plurality of polytopic organic linkers has the formula:

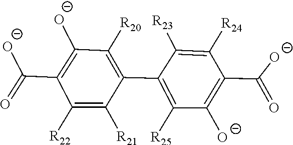

where R₂₀, R₂₁, R₂₂, R₂₃, R₂₄, and R₂₅ are each independently selected from H, halogen, hydroxyl, methyl, or halogen substituted methyl. In some embodiments, R₂₀, R₁₁, R₂₂, R₂₃, R₂₄, and R₂₅ are each hydrogen. In some embodiments, the polytopic organic linker is unprotonated as illustrated, partially protonated, or fully protonated in the solution.

In some embodiments, the polytopic organic linker is 2,5-dioxido-1,4-benzenedicarboxylate (dobdc⁴⁻), 4,6-dioxido-1,3-benzenedicarboxylate (m-dobdc⁴⁻), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), dioxidobiphenyl-4,4′-dicarboxylate (para-carboxylate-dobpdc-, also referred to as pc-dobpdc⁴⁻), 2,5-dioxidobenzene-1,4-dicarboxylate (dobdc⁴⁻), 1,3,5-benzenetristetrazolate (BTT), 1,3,5-benzenetristriazolate (BTTri), 1,3,5-benzenetrispyrazolate (BTP), or 1,3,5-benzenetriscarboxylate (BTC).

In some embodiments the compound of formula MₙXₘ is a magnesium(II) metal salt, a manganese(II) metal salt, an iron(II) metal salt, a cobalt (II) metal salt, a nickel(II) metal salt, a zinc(II) metal salt, or a cadmium(II) metal salt. In some embodiments, the metal salt is cobalt(II) nitrate, cobalt(II) chloride, cobalt(II) acetate, cobalt(II) sulfate, cobalt(II) iodide, cobalt(II) bromide, cobalt(II) trifluorosulfonate, cobalt(II) tetrafluoroborate, cobalt(II) oxide, cobalt(II) carbonate, cobalt(II) hydroxide, cobalt(II) hydroxycarbonate, mixed-halide cobalt(II), cobalt(II) acetylacetonate, cobalt(II) formate, cobalt(II) perchlorate or a halogenated derivative thereof. In some embodiments, the basic anion is formate or acetate. In some embodiments, the basic anion is sulfate, bromide, iodide, or triflurosulfonate and the reacting is in the presence of a buffer devoid of metal coordinating functionality.

### IV. Exemplary Synthetic Schemes

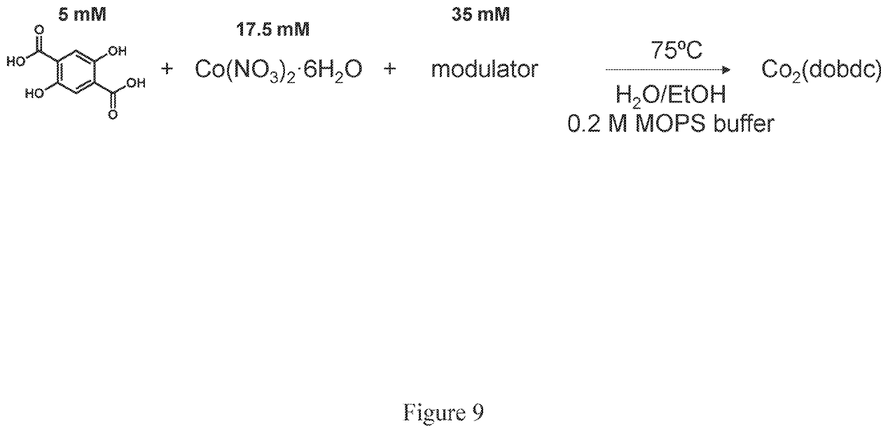

In the present disclosure, crystalline metal-organic frameworks comprising a plurality of metal cations and a plurality of polytopic organic linkers are synthesized, where each polytopic organic linker in the plurality of polytopic organic linkers is connected to two or more metal cations in the plurality of metal cations, and the crystalline metal-organic framework is characterized by one or more pore channels. In some such embodiments, the plurality of polytopic organic linkers are reacted with one or more compounds of formula MₙXₘ, where each M is independently cationic Be. Mg, Ca. Ti, V, Cr, Mn, Fe, Co, Ni, Cu, Zn, Zr, Nb, Mo, Ru, Rh, Pd, Cd, or Hf, X is a basic anion (e.g., formate, acetate, sulfate, bromide, iodide, or triflurosulfonate, etc.), and n is a positive integer (e.g., 1, 2, etc.) and m is a positive integer (e.g., 1, 2, etc.). The reacting is in the presence of a modulator such as a salicylate, salicylamide, 1,3-phthalate, 3-hydroxybenzoate or a salt (e.g., sodium, potassium cesium) thereof, or a mixture thereof. In this way, the crystalline morphology of the resultant metal-organic framework crystals (e.g., in one or more crystallographic directions or a linear combination thereof) is controlled. For example, in some embodiments, crystal growth of the metal-organic framework results in crystals that have needle, disk, or rice grain shapes. FIG. 9 illustrates example concentrations for the metal cation, polytopic organic linkers, and modulator, as well as example buffer and pH, solvent, and reaction temperature used in accordance with the present disclosure to produce Co₂(dobdc) crystals.

In some embodiments, the plurality of polytopic organic linkers is present in the solution at a concentration of between 1 mM and 50 mM prior to the reacting, the one or more compounds of formula MₙXₘ is present in the solution at a concentration of between 5 nM and 100 nM prior to the reacting, and the modulator is present in the solution at a concentration of between 15 nM and 100 nM prior to the reacting.

In some embodiments, the plurality of polytopic organic linkers is present in the solution at a concentration of at least 0.1, at least 0.5, at least 1, at least 5, at least 10, at least 15, at least 20, at least 25, at least 30, at least 35, at least 40, at least 45, at least 50, at least 60, at least 70, at least 80, at least 90, at least 100, at least 150, at least 200, at least 300, at least 400, at least 500, at least 600, at least 700, at least 800, or at least 900 mM. In some embodiments, the plurality of polytopic organic linkers is present in the solution at a concentration of at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, or at least 500 M. In some embodiments, the plurality of polytopic organic linkers is present in the solution at a concentration of no more than 800, no more than 500, no more than 100, no more than 80, no more than 50, no more than 20, no more than 10, no more than 5, or no more than 1 M. In some embodiments, the plurality of polytopic organic linkers is present in the solution at a concentration of no more than 800, no more than 500, no more than 100, no more than 80, no more than 50, no more than 20, no more than 10, no more than 5, or no more than 1 mM. In some embodiments, the plurality of polytopic organic linkers is present in the solution at a concentration of from 0.5 mM to 10 mM, from 1 mM to 200 mM, from 50 mM to 500 mM, from 200 mM to 1 M, from 1 M to 50 M, from 10 M to 200 M, or from 50 mM to 10 M. In some embodiments, the plurality of polytopic organic linkers is present in the solution at a concentration that falls within another range starting no lower than 0.1 mM and ending no higher than 800 M.

In some embodiments, the one or more compounds of formula MₙXₘ is present in the solution at a concentration of at least 0.1, at least 0.5, at least 1, at least 5, at least 10, at least 15, at least 20, at least 25, at least 30, at least 35, at least 40, at least 45, at least 50, at least 60, at least 70, at least 80, at least 90, at least 100, at least 150, at least 200, at least 300, at least 400, at least 500, at least 600, at least 700, at least 800, or at least 900 nM. In some embodiments, the one or more compounds of formula MₙXₘ is present in the solution at a concentration of at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, or at least 500 mM. In some embodiments, the one or more compounds of formula MₙXₘ is present in the solution at a concentration of no more than 1000, no more than 500, no more than 100, no more than 80, no more than 50, no more than 20, no more than 10, no more than 5, or no more than 1 mM. In some embodiments, the one or more compounds of formula MₙXₘ is present in the solution at a concentration of no more than 800, no more than 500, no more than 100, no more than 80, no more than 50, no more than 20, no more than 10, no more than 5, or no more than 1 nM. In some embodiments, the one or more compounds of formula MₙXₘ is present in the solution at a concentration of from 0.5 nM to 10 nM, from 1 nM to 200 nM, from 50 nM to 500 nM, from 200 nM to 1 mM, from 1 mM to 50 mM, from 10 mM to 200 mM, or from 50 nM to 10 mM. In some embodiments, the one or more compounds of formula MₙXₘ is present in the solution at a concentration that falls within another range starting no lower than 0.1 nM and ending no higher than 1 M.

In some embodiments, the modulator is present in the solution at a concentration of at least 0.1, at least 0.5, at least 1, at least 5, at least 10, at least 15, at least 20, at least 25, at least 30, at least 35, at least 40, at least 45, at least 50, at least 60, at least 70, at least 80, at least 90, at least 100, at least 150, at least 200, at least 300, at least 400, at least 500, at least 600, at least 700, at least 800, or at least 900 nM. In some embodiments, the modulator is present in the solution at a concentration of at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, or at least 500 mM. In some embodiments, the modulator is present in the solution at a concentration of no more than 1000, no more than 500, no more than 100, no more than 80, no more than 50, no more than 20, no more than 10, no more than 5, or no more than 1 mM. In some embodiments, the modulator is present in the solution at a concentration of no more than 800, no more than 500, no more than 100, no more than 80, no more than 50, no more than 20, no more than 10, no more than 5, or no more than 1 nM. In some embodiments, the modulator is present in the solution at a concentration of from 0.5 nM to 10 nM, from 1 nM to 200 nM, from 50 nM to 500 nM, from 200 nM to 1 mM, from 1 mM to 50 mM, from 10 mM to 200 mM, or from 50 nM to 10 mM. In some embodiments, the modulator is present in the solution at a concentration that falls within another range starting no lower than 0.1 nM and ending no higher than 1 M.

In some embodiments n is 1 or 2 and m is 1 or 2. In some embodiments, n is 1 and m either 1 or 2. In some embodiments, m is 2 or greater.

In some embodiments, each polytopic organic linker in the plurality of polytopic organic linkers is connected to two metal cations in the plurality of metal cations.

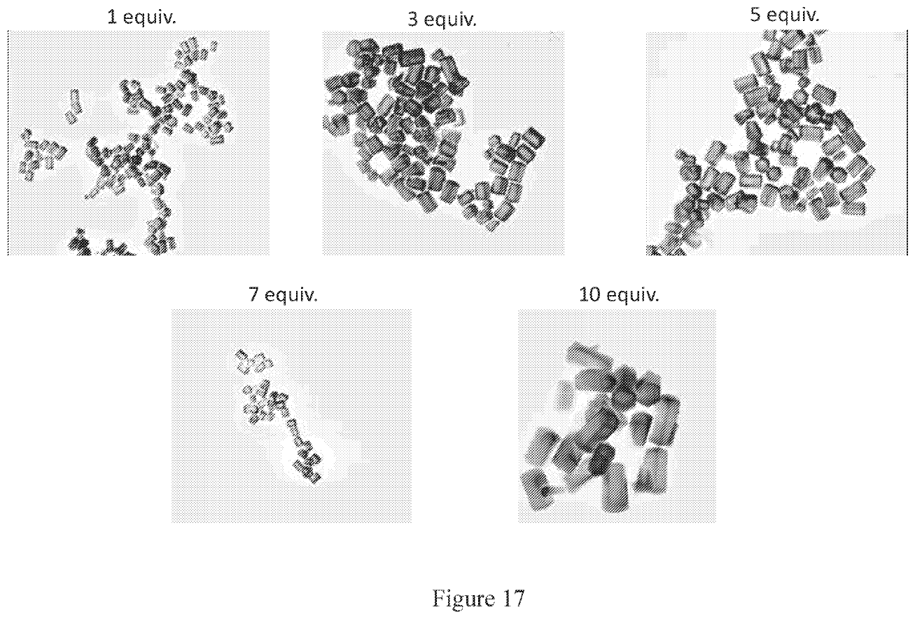

FIG. 17 illustrates consistently smaller crystals at 10 equivalents of 3-hydroxybenzoate than 1 equivalent, but crystal size does not monotonically decrease with modulator concentration, with the same trends observed for salicylamide, phthalate, and bromosalicylamide, in accordance with an embodiment of the present disclosure. Accordingly, in some embodiments, the number of equivalents of the polytopic organic linker relative to the number of equivalents of the modulator is varied in order to optimize MOF crystal size and morphology. For instance, in some embodiments there is one equivalent of the polytopic organic linker to between 0.5 and 20 equivalents of the modulator in the solution prior to the reacting. In some embodiments, there is one equivalent of the polytopic organic linker to between 1 and 15 equivalents of the modulator in the solution prior to the reacting. In some embodiments, the ratio of the number of equivalents of the polytopic organic linker to the number of equivalents of the modulator is no more than 1:0.001, no more than 1:0.01, no more than 1:0.1, no more than 1:0.5, no more than 1:1, no more than 1:2, no more than 1:3, no more than 1:4, no more than 1:5, no more than 1:6, no more than 1:7, no more than 1:8, no more than 1:9, no more than 1:10, no more than 1:15, no more than 1:20, no more than 1:30, no more than 1:40, no more than 1:50, no more than 1:60, no more than 1:70, no more than 1:80, no more than 1:90, or no more than 1:100. In some embodiments, the ratio of the number of equivalents of the polytopic organic linker to the number of equivalents of the modulator is at least 1:200, at least 1:100, at least 1:80, at least 1:50, at least 1:40, at least 1:20, at least 1:10, at least 1:5, at least 1:2, or at least 1:1. In some embodiments, the ratio of the number of equivalents of the polytopic organic linker to the number of equivalents of the modulator is from 1:5 to 1:0.1, from 1:50 to 1:1, or from 1:30 to 1:2. In some embodiments, the ratio of the number of equivalents of the polytopic organic linker to the number of equivalents of the modulator falls within another range starting no lower than 1:200 and ending no higher than 1:0.001.

In some embodiments, the modulator has the formula:

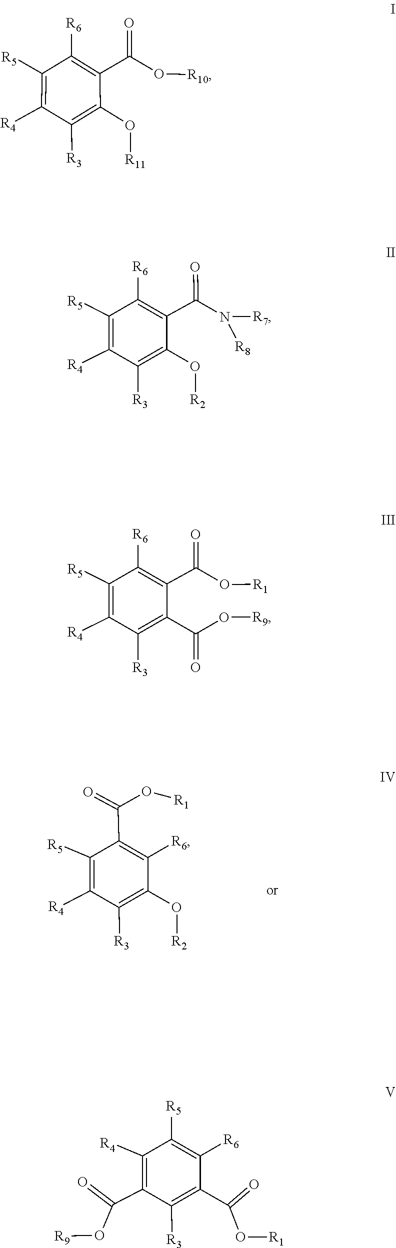

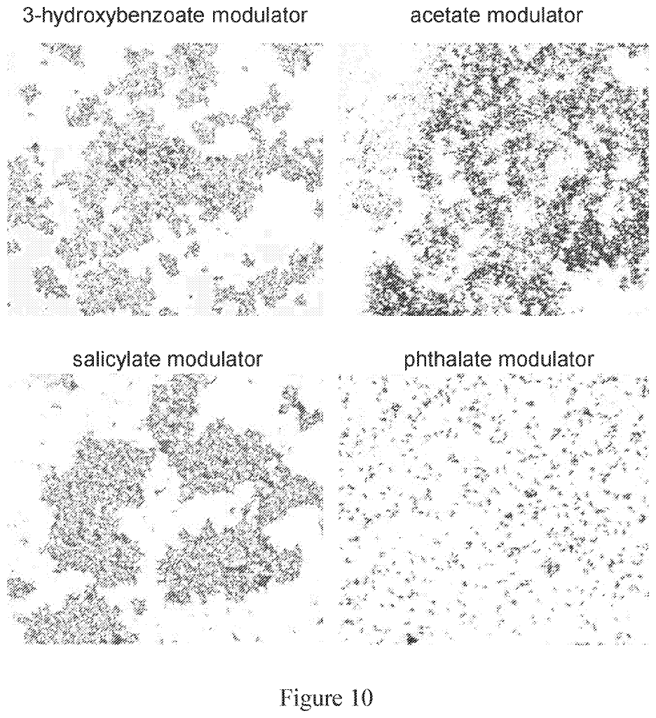

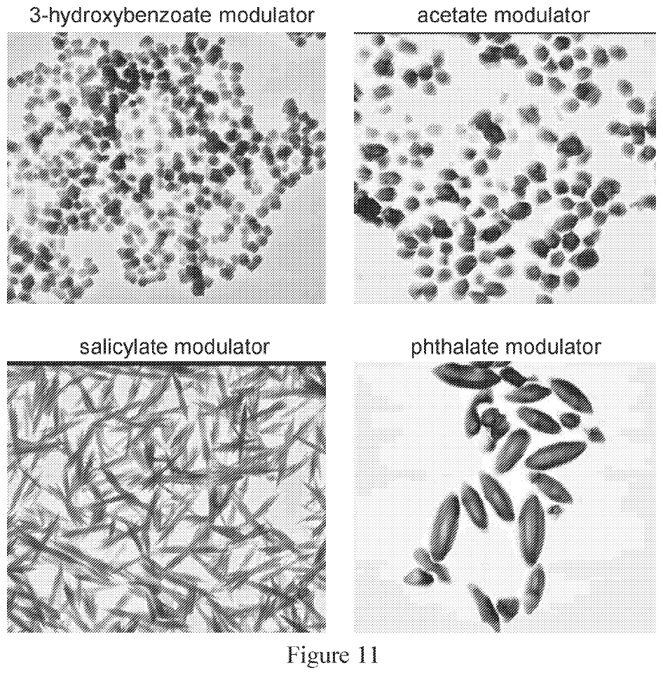

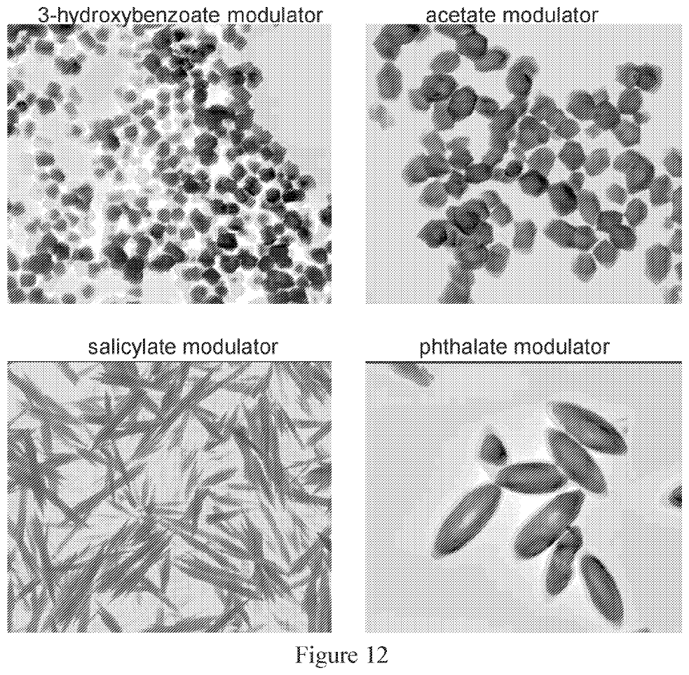

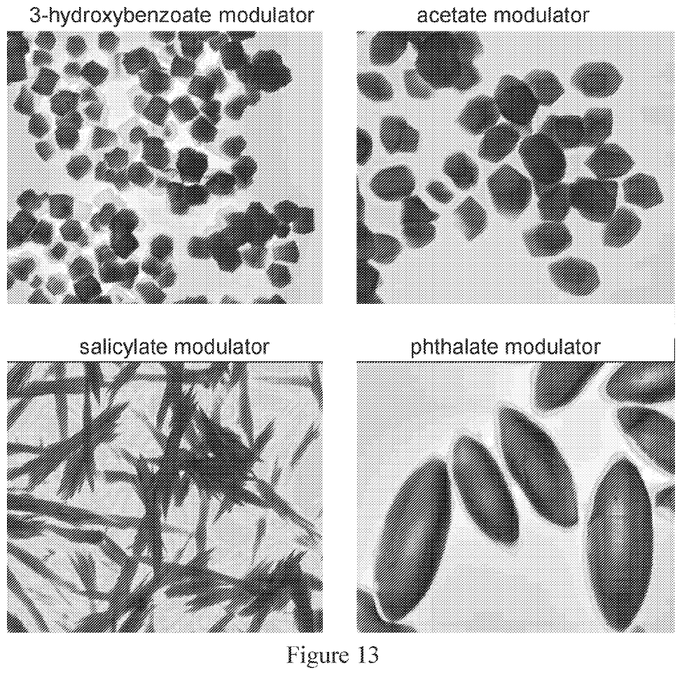

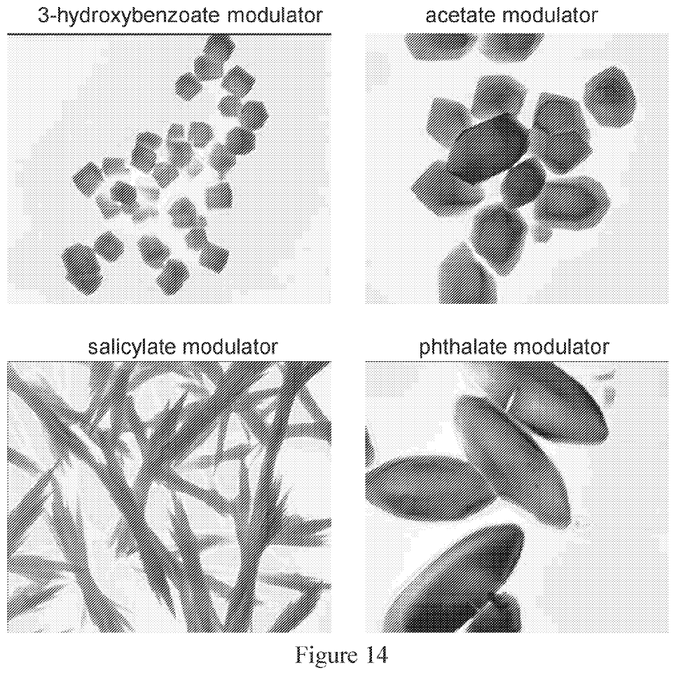

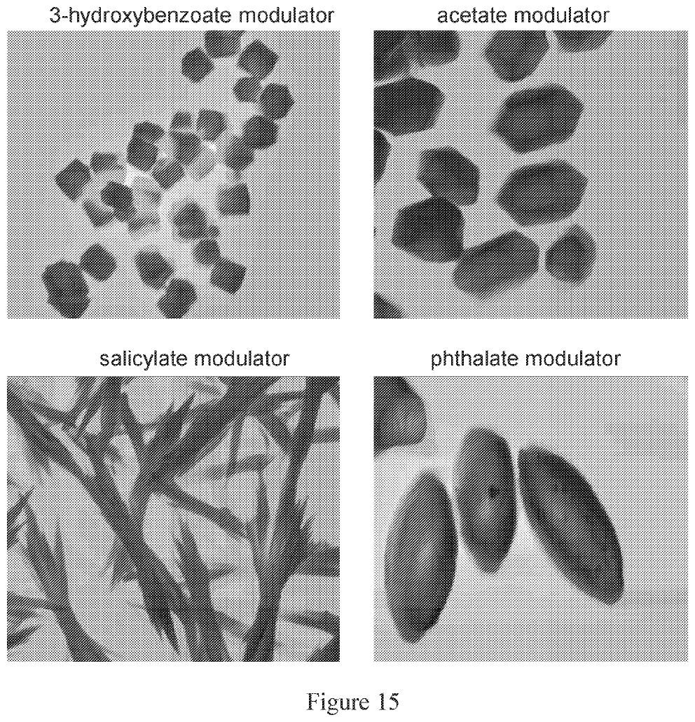

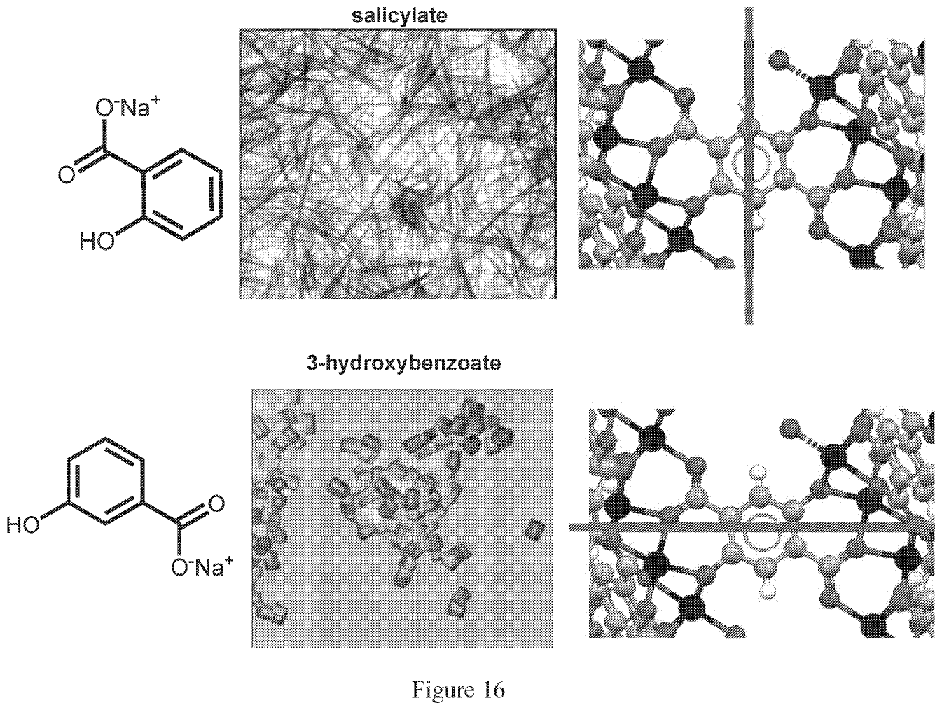

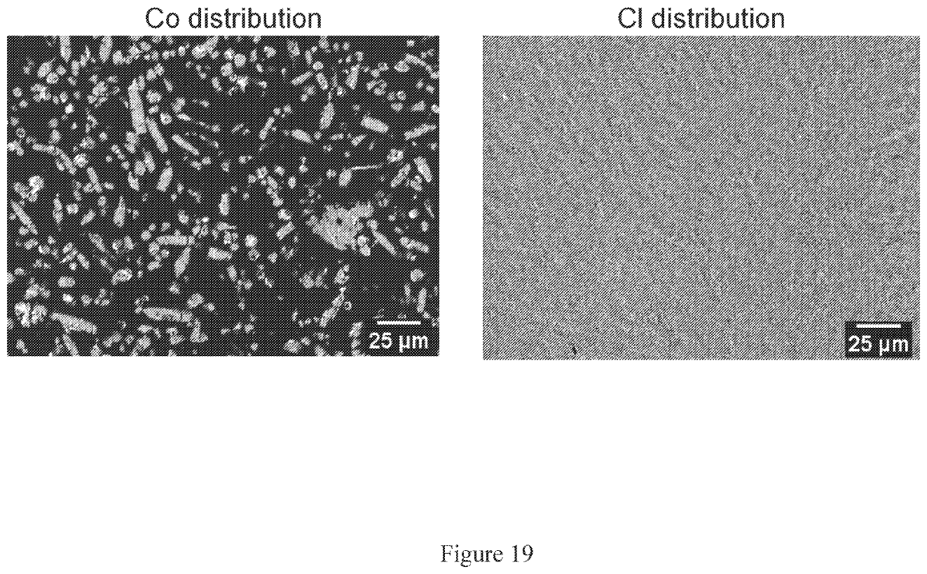

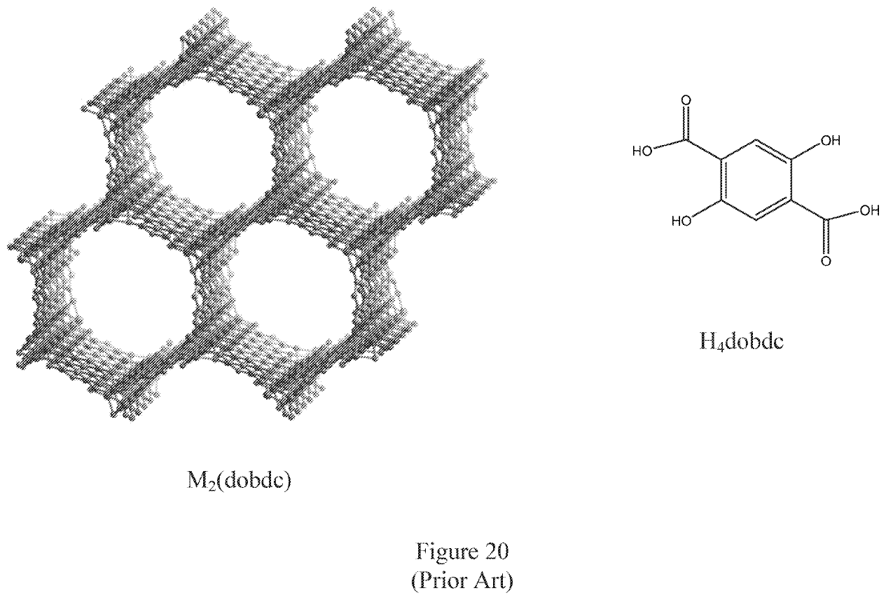

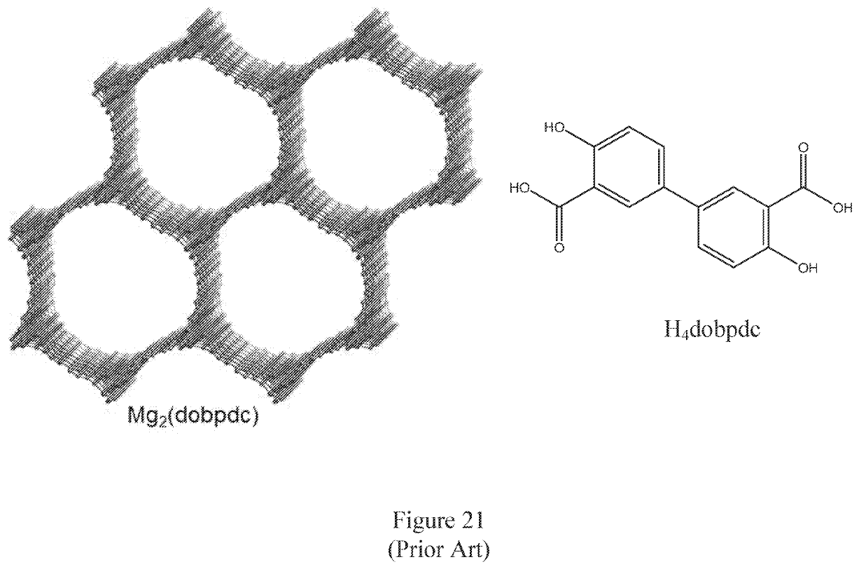

or a salt (e.g., sodium, potassium cesium) thereof or a mixture thereof (e.g. a mixture of any two, three, four, five, six, seven, eight, nine or ten modulators each independently having formula I, II, III, IV, or V) where R₁, R₂, R₇, R₈, R₉, R₁₀ and R₁₁ are each independently selected from hydrogen, halogen, substituted or unsubstituted alkyl, substituted or unsubstituted heteroalkyl, substituted or unsubstituted aryl, substituted or unsubstituted heteroaryl, or substituted or unsubstituted heterocycloalkyl. R₃, R₄, R₅, and R₅ are each independently selected from H, halogen, hydroxyl, methyl, or halogen substituted methyl, and where R₁₀ and R₁₁ are not each hydrogen. For example, FIG. 10 illustrates, in clockwise fashion, Co₂(dobdc) crystals at 350× magnification grown in the presence of the modulators 3-hydroxybenzoate, acetate, salicylate, and phthalate in accordance with embodiments of the present disclosure. FIGS. 11 through 15 illustrate in clockwise fashion Co₂(dobdc) crystals grown in the presence of the modulators 3-hydroxybenzoate, acetate, salicylate, and phthalate at various levels of magnification. FIG. 16 illustrates Co₂(dobdc) crystals grown in the presence of the modulator salicylate at 1504× magnification (top), and 3-hydroxybenzoate at 2000× magnification (bottom). FIG. 19 illustrates how modulator incorporation into the metal-organic framework is extremely low, in the case of trichloroacetate-modulated metal-organic framework crystallization, it is 83 ppm by elemental analysis (left panel) and it is not visible by energy dispersive X-ray spectroscopy, in accordance with an embodiment of the present disclosure.

In some such embodiments, R₃, R₄, R₅, and R₆ are each independently a substituted or unsubstituted linear or branched alkyl having between one and ten carbon atoms.

In some such embodiments, R₃, R₄, R₅, and R₆ are each hydrogen.

In some embodiments, the solution contains a single modulator and this single modulator has formula IV in which R₁ and R₂ are each hydrogen. In some such embodiments, R₃, R₄, R₅, and R₆ are each hydrogen.

In some embodiments, the solution contains a single modulator and this single modulator has formula II in which R₂, R₇ and R₈ are each hydrogen. In some such embodiments, R₃, R₄, R₅, and R₆ are each hydrogen.

In some embodiments, the solution contains a single modulator, and this single modulator has formula III or V in which R₁ and R₉ are each hydrogen. In some such embodiments, R₃, R₄, R₅, and R₆ are each hydrogen.

In some embodiments, a compound in the one or more compounds of formula MₙXₘ is a magnesium(II) metal salt, a manganese(II) metal salt, an iron(II) metal salt, a cobalt (11) metal salt, a nickle(II) metal salt, a zinc(II) metal salt, or a cadmium(II) metal salt.

In some embodiments, the one or more compounds is a single compound and this single compound is of formula MₙXₘ where MₙXₘ is a magnesium(II) metal salt, a manganese(II) metal salt, an iron(II) metal salt, a cobalt (II) metal salt, a nickle(II) metal salt, a zinc(II) metal salt, or a cadmium(II) metal salt.

In some embodiments, the reacting is performed with a buffer devoid of metal coordinating functionality (non-coordinating buffers). The disclosed non-coordinating buffers form their genesis in the work of Good et al., 1966, Biochem. 5(2), 467, as further developed by Kandegedara and Rorabacher, 1999, Anal. Chem. 71, 3140, each of which is hereby incorporated by reference. In some embodiments, the buffer devoid of metal coordinating functionality is PIPES, PIPPS, PIPBS, DEPP, DESPEN, MES, TEEN, PIPES, MOBS, DESPEN, or TEMN. See Kandegedara and Rorabacher, 1999, Anal. Chem. 71, 3140. In some embodiments the buffer devoid of metal coordinating functionality is an alkyl or alkylsulfonate derivative of morpholine, piperazine, ethylenediamine, or methylenediamine.

There are reports of surface- or epitaxially-grown metal-organic frameworks. See Heinke et al., 2016, SURMOFs: Liquid-Phase Epitaxy of Metal-Organic Frameworks on Surfaces, in The Chemistry of Metal-Organic Frameworks: Synthesis, Characterization, and Applications (ed S. Kaskel), Wiley-VCH Verlag GmbH & Co. KGaA, Weinheim, Germany. doi: 10.1002/9783527693078, ch17, which is hereby incorporated by reference. In general, the synthetic procedure for surface- or epitaxially-grown metal-organic frameworks seeks to functionalize a surface and cause nucleation directly on the surface, generally with layer-by-layer growth. The strategy is normally contrasted with solvothermal or hydrothermal synthesis, whereby the products form at elevated temperature in solution. While the surface is of concern in epitaxially-grown metal-organic frameworks, little attention has been paid to the role of surfaces in solvothermal and hydrothermal reactions. The process of silanization is a well-established technique to impart functionality or hydrophobicity to the surface of glassware. See Seed, 2001, “Silanizing Glassware”, Current Protocols in Cell Biology 8:3E:A.3E.1-A.3E.2 and Plueddemann, 1991, “Chemistry of Silane Coupling Agents.” In: Silane Coupling Agents, Springer, Boston, MA, each of which is incorporated by reference. However, the effect of using different hydrophobic surfaces during solvothermal or hydrothermal syntheses of metal-organic frameworks has not been studied for phase selection or for morphological control.

Dramatically different methods of synthesis have been found to lead to different metal-organic framework morphologies. For example, microwave heating has been found to lead to different crystallite size than solvothermal growth. See Stock and Biswas, 2012, Chem. Rev. 112, 933, which is hereby incorporated by reference. However, within hydrothermal synthesis, the effect of oil bath heating versus oven heating has only been studied in limited conditions for the framework UiO-66 and In-MIL-68, and not at all for synthesis of the frameworks M₂(dobdc) or M₂(dobpdc). See Lee et al., 2017, Cryst. Eng. Comm. 19, 426, which is hereby incorporated by reference.

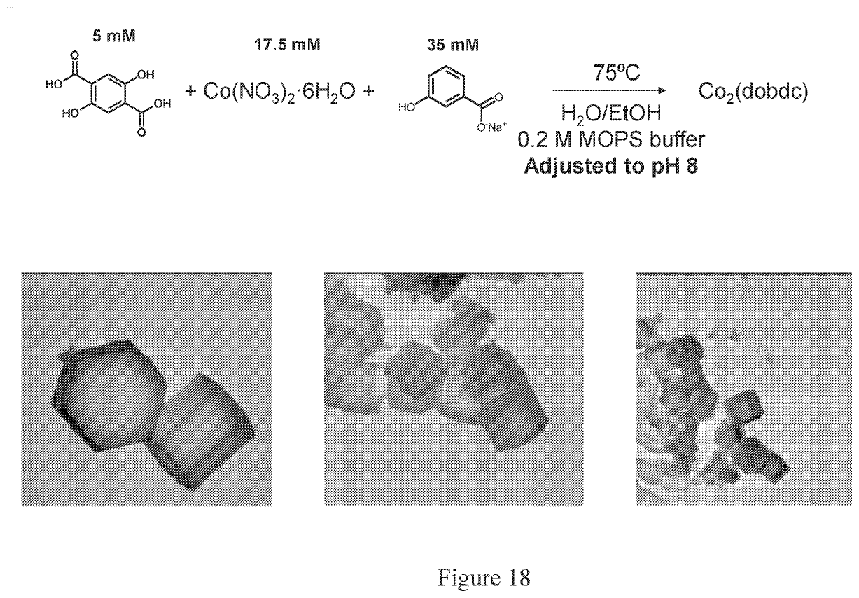

FIG. 18 illustrates how higher pH leads to lower aspect ratio crystals. Accordingly, in some embodiments, the pH of the solution is between 6.5 and 8.5. In some embodiments, the pH of the solution is between 7.0 and 8.0. In some embodiments, the pH of the solution is at least 2, at least 3, at least 4, at least 4.5, at least 5, at least 5.5, at least 6, at least 6.5, at least 7, at least 7.5, at least 8, at least 8.5, at least 9, or at least 9.5. In some embodiments, the pH of the solution is no more than 13, no more than 12, no more than 11, no more than 10.5, no more than 10, no more than 9.5, no more than 9, no more than 8.5, no more than 8, no more than 7.5, no more than 7, or no more than 6.5. In some embodiments, the pH of the solution is from 6 to 9, from 6 to 10, from 5 to 8, from 7 to 9, or from 6 to 8. In some embodiments, the pH of the solution falls within another range starting no lower than 1 and ending no higher than 14.

One aspect of the present disclosure provides for the synthesis of metal-organic frameworks in the presence of a non-coordinating buffer or non-coordinating base, with controlled heating and/or reaction vessel functionalization. The strategy of employing non-coordinating buffers, acids, or bases allows for controlled deprotonation of the ligand at a wide range of pH values without interfering with the coordination equilibria desired to effect a certain crystal morphology. Using these tools, the pH can be set independently from the solvent/ligand/counterion coordination during growth. Further, by controlling the pH without relying on solvent decomposition, precise control over what coordinating agents are available in solution is provided. Judicious choice of additive or metal counterion allows preferential coordination during growth to some crystal facets, slowing growth in that direction. While modulation has been known to affect particle size and stability, selective attachment to one end of the crystal structure is non-obvious. Use of a stronger coordinating agent selectively slows growth along the direction of the pore channel. See International Patent Publication Number WO2020/068996 entitled “Metal-Organic Framework Phase and Crystallite Shape Control,” which is hereby incorporated by reference.

In some embodiments, M is cationic Fe, Co or Zn. In some embodiments, n is 2, 3, or 4. In some embodiments, n is 2. In some embodiments, n is 5 or 6.

In some embodiments, the pKa value of the basic anion is below 3.5. In some embodiments the pKa value of the basic anion is above 3.5. In some embodiments, the pKa of the anion is above the lowest pKa value of the polytopic organic linker.

As an example the crystalline MOF material may be formed in accordance with synthetic methods of the present disclosure from a variety of cobalt(II) salts including cobalt(II) nitrate, cobalt(II) chloride, cobalt(II) acetate, cobalt(II) sulfate, cobalt(II) iodide, cobalt(II) bromide, cobalt(II) trifluorosulfonate, cobalt(II) tetrafluoroborate, cobalt(II) oxide, cobalt(II) carbonate, cobalt(II) hydroxide, cobalt(II) hydroxycarbonate, mixed-halide cobalt(II), cobalt(II) acetylacetonate, cobalt(II) formate, cobalt(II) perchlorate, or halogenated derivatives thereof.

In some embodiments, the plurality of polytopic organic linkers are at a concentration of between 1 mM and 1 M, at a concentration of between 3 mM and 0.5 M or at a concentration of between 4 mM and 250 mM in the solution prior to initiating the reacting.

In some embodiments, each polytopic organic linker in the plurality of polytopic organic linkers has the formula:

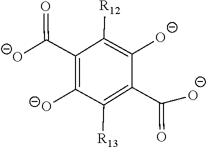

where, R₁₂ and R₁₃ are each independently selected from H, halogen, hydroxyl, methyl, or halogen substituted methyl. In some embodiments, R₁₃ and R₁₃ are each hydrogen.

In some embodiments, each polytopic organic linker in the plurality of polytopic organic linkers has the formula:

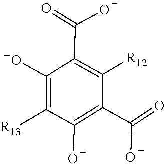

where R₁₂ and R₁₃ are each independently selected from H, halogen, hydroxyl, methyl, or halogen substituted methyl. In some embodiments, R₁₂ and R₁₃ are each hydrogen.

In some embodiments, each polytopic organic linker in the plurality of polytopic organic linkers has the formula:

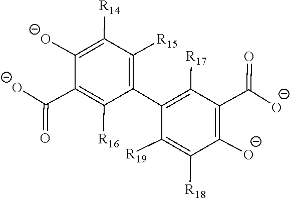

where R₁₄, R₁₅, R₁₆, R₁₇, R₁₈, and R₁₉ are each independently selected from H, halogen, hydroxyl, methyl, or halogen substituted methyl. In some embodiments R₁₄, R₁₅, R₁₆, R₁₇, R₁₈, and R₁₉ are each hydrogen.

In some embodiments, each polytopic organic linker in the plurality of polytopic organic linkers has the formula:

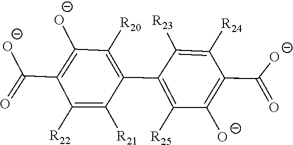

where R₂₀, R₂₁, R₂₂, R₂₃, R₂₄, and R₂₅ are each independently selected from H, halogen, hydroxyl, methyl, or halogen substituted methyl. In some such embodiments R₂₀, R₂₁, R₂₂, R₂₃, R₂₄, and R₂₅ are each hydrogen.

In some embodiments each polytopic organic linker in the plurality of polytopic organic linkers is: 2,5-dioxido-1,4-benzenedicarboxylate (dobdc⁴⁻), 4,6-dioxido-1,3-benzenedicarboxylate (m-dobdc⁴⁻), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), dioxidobiphenyl-4,4′-dicarboxylate (para-carboxylate-dobpdc⁴⁻, also referred to as pc-dobpdc⁴⁻), 2,5-dioxidobenzene-1,4-dicarboxylate (dobdc⁴⁻), 1,3,5-benzenetristetrazolate (BTT), 1,3,5-benzenetristriazolate (BTTri), 1,3,5-benzenetrispyrazolate (BTP), or 1,3,5-benzenetriscarboxylate (BTC).

In some embodiments, each polytopic organic linker in the plurality of polytopic organic linkers is 2,5-dioxido-1,4-benzenedicarboxylate (dobdc⁴).

In some embodiments, the reacting is performed at a temperature greater than 60° C. In some embodiments, the reacting is performed at a temperature between 70° C. and 80° C.

In some embodiments, the salt of the modulator is a sodium salt, a potassium salt, or a cesium salt.

In some embodiments the reacting occurs at a temperature less than 30° C. for two to three days, at a temperature less than 40° C. for less than one or two days, at a temperature less than 45° C. between 10 and 25 hours, at a temperature less than 50° C. for at least eleven hours, at a temperature less than 60° C. for at least eight hours, at a temperature less than 70° C. for at least two hours, at a temperature less than 80° C. for at least 30 minutes, or at a temperature less than 90° C. for at least 10 minutes.

In some embodiments the reacting occurs at a temperature between 30° C. and 50° C. for two to three days, at a temperature between 35° C. and 55° C. for between one and three days, at a temperature between 40° C. and 60° C. for between 10 and 25 hours, at a temperature between 45° C. and 70° C. for at least eleven hours, at a temperature of between 45° C. and 70° C. for at least eight hours, at a temperature between 60° C. and 80° C. for at least two hours, at a temperature between 70° C. and 90° C. for at least 30 minutes, or at a temperature between 80° C. and 100° C. for at least 10 minutes.

In some embodiments the reacting occurs at a temperature greater than 60° C. for at least eight hours, at a temperature greater than 60° C. for at least nine hours, at a temperature greater than 60° C. for at least ten hours, at a temperature greater than 60° C. for at least eleven hours, at a temperature greater than 60° C. for at least twelve hours, at a temperature greater than 60° C. for at least thirteen hours, at a temperature greater than 60° C. for at least fourteen hours, or at a temperature greater than 60° C. for at least fifteen hours.

In some embodiments of the reacting occurs at a temperature greater than 62° C. for at least eight hours, at a temperature greater than 62° C. for at least nine hours, at a temperature greater than 62° C. for at least ten hours, at a temperature greater than 62° C. for at least eleven hours, at a temperature greater than 62° C. for at least twelve hours, at a temperature greater than 62° C. for at least thirteen hours, at a temperature greater than 62° C. for at least fourteen hours, or at a temperature greater than 62° C. for at least fifteen hours.

In some embodiments the reacting occurs at a temperature greater than 64° C. for at least eight hours, at a temperature greater than 64° C. for at least nine hours, at a temperature greater than 64° C. for at least ten hours, at a temperature greater than 64° C. for at least eleven hours, at a temperature greater than 64° C. for at least twelve hours, at a temperature greater than 64° C. for at least thirteen hours, at a temperature greater than 64° C. for at least fourteen hours, or at a temperature greater than 64° C. for at least fifteen hours.

In some embodiments the reacting occurs at a temperature greater than 66° C. for at least eight hours, at a temperature greater than 66° C. for at least nine hours, at a temperature greater than 66° C. for at least ten hours, at a temperature greater than 66° C. for at least eleven hours, at a temperature greater than 66° C. for at least twelve hours, at a temperature greater than 66° C. for at least thirteen hours, at a temperature greater than 66° C. for at least fourteen hours, or at a temperature greater than 66° C. for at least twenty hours.

In some embodiments the reacting occurs at a temperature greater than 68° C. for at least eight hours, at a temperature greater than 68° C. for at least nine hours, at a temperature greater than 68° C. for at least ten hours, at a temperature greater than 68° C. for at least eleven hours, at a temperature greater than 68° C. for at least twelve hours, at a temperature greater than 68° C. for at least thirteen hours, at a temperature greater than 68° C. for at least fourteen hours, or at a temperature greater than 68° C. for at least twenty hours.

In some embodiments the reacting occurs at a temperature greater than 70° C. for at least eight hours, at a temperature greater than 70° C. for at least nine hours, at a temperature greater than 70° C. for at least ten hours, at a temperature greater than 70° C. for at least eleven hours, at a temperature greater than 70° C. for at least twelve hours, at a temperature greater than 70° C. for at least thirteen hours, at a temperature greater than 70° C. for at least fourteen hours, or at a temperature greater than 70° C. for at least twenty hours.

In some embodiments the reacting occurs at a temperature greater than 72° C. for at least eight hours, at a temperature greater than 72° C. for at least nine hours, at a temperature greater than 72° C. for at least ten hours, at a temperature greater than 72° C. for at least eleven hours, at a temperature greater than 72° C. for at least twelve hours, at a temperature greater than 72° C. for at least thirteen hours, at a temperature greater than 72° C. for at least fourteen hours, or at a temperature greater than 72° C. for at least twenty hours.

In some embodiments the reacting occurs at a temperature greater than 74° C. for at least eight hours, at a temperature greater than 74° C. for at least nine hours, at a temperature greater than 74° C. for at least ten hours, at a temperature greater than 74° C. for at least eleven hours, at a temperature greater than 74° C. for at least twelve hours, at a temperature greater than 74° C. for at least thirteen hours, at a temperature greater than 74° C. for at least fourteen hours, or at a temperature greater than 74° C. for at least twenty hours.

In some embodiments, the reacting occurs at a temperature greater than 25° C. for at least one hour. In some embodiments, the reacting is performed at a temperature greater than 25° C. for at least eight hours.

In some embodiments, the compound of formula MₙXₘ is at a concentration of between 5 mM and 1 M, between 10 mM and 0.5 M, or between 15 mM and 250 mM in the solution prior to the reacting.

In some embodiments a buffer devoid of metal coordinating functionality is used in the reacting. In some embodiments, this buffer is PIPES, PIPPS, PIPBS, DEPP, DESPEN, MES, TEEN, PIPES, MOBS, DESPEN, or TEMN. In some embodiments a buffer devoid of metal coordinating functionality is used and this buffer is an alkyl or alkylsulfonate derivative of morpholine, piperazine, ethylenediamine, or methylenediamine. See Kandegedara and Rorabacher, 1999, Anal. Chem. 71, 3140, which is hereby incorporated by reference.

In some embodiments a buffer devoid of metal coordinating functionality is buffered to a concentration of between 0.05 M and 0.5 M, between 0.10 M and 0.4 M, between 0.15 M and 0.30 M, or between 0.18 M and 0.22 M in the solution prior to the reacting. In some embodiments, a buffer devoid of metal coordinating functionality is used in the solution and is buffered to a pH of below 5.0, between 5.0 and 6.0, between 6.0 and 7.0, between 7.0 and 8.0, or above 8.0.

In some embodiments a buffer devoid of metal coordinating functionality is present in the solution and this buffer does not measurably interact with or ligate to the metal cations of the crystalline metal-organic framework.

In some embodiments the solution comprises polar protic solvent or a mixture of polar protic solvents. In some embodiments, the solution comprises an ethanol:water solvent mixture. In some embodiments, the solution comprises an x:y mixture of ethanol and water, where x and y are independent separate positive integers. In some embodiments x is 1 and y is 1. In some embodiments x is 2 and y is 1. In some embodiments, y is 2 and x is 1. In some embodiments, x ranges between 0.5 and 5 and y ranges between 5 and 0.5. In some embodiments, the reacting occurs in a 1:1 ethanol:H₂O solvent. In some embodiments the reacting occurs in t-butanol, n-propanol, ethanol, methanol, acetic acid, water, N,N-dimethylformamide or a mixture thereof.

Heating apparatus. During hydrothermal synthesis, the method of providing heat to identical synthesis reactions affects the size, morphology, and dispersity of metal-organic framework samples. The dispersity and size of MOF crystals can be controlled via changes in heating apparatus such as an oil bath, oven, or metal bead bath. For otherwise identical synthetic conditions, an oil bath is found to improve crystallite dispersity for syntheses done in unsilanized glassware in some embodiments. Examples of metal bead baths are the metal bead bath product line of Lab Armor (Cornelius, Oregon), including the Lab Armor 74300-714 Waterless Bead Bath, 14 L capacity. Some embodiments of the present disclosure further specify that the disclosed reacting be done using specific forms or heat sources as disclosed below.

Use of an oil bath. In some embodiments, the disclosed reacting step is performed in unsilanized glassware using an oil bath.

In one example embodiment of the present disclosure, the crystalline metal-organic framework is formed in accordance with the disclosed synthetic schemes by solubilizing the polytopic organic linker in a first polar protic solvent. Separately, the compound of formula MₙXₘ is dissolved in a second polar protic solvent. In some embodiments the first polar protic solvent and the second polar protic solvent are the same. In some embodiments the first polar protic solvent and the second polar protic solvent are the different. The reaction commences upon the mixing of the two solutions, for instance in a 250 mL three-neck, round bottom flask with a Dimroth condenser at 15° C. In some embodiments, the mixed solution is refluxed inside the round bottom flask that is placed in an oil bath at an elevated temperature (e.g., greater than 60° C.) for a period of time (e.g., greater than 10 hours) under agitation (e.g., 300 rpm). At the end of the reaction time, the solution is cooled to room temperature (e.g., by removal from the oil bath or by cooling the oil bath).

In another embodiment of the present disclosure, the crystalline metal-organic framework is formed by solubilizing the polytopic organic linker in a first polar protic solvent. Separately, the compound of formula MₙXₘ is dissolved in a second polar protic solvent. In some embodiments the first polar protic solvent and the second polar protic solvent are the same. In some embodiments the first polar protic solvent and the second polar protic solvent are the different. The reaction commences upon the instant mixing of the two solutions, for instance in a 250 mL three-neck, round bottom flask with a Dimroth condenser at 15° C. In some embodiments, the mixed solution is refluxed inside the round bottom flask that is placed in an oil bath at an elevated temperature (e.g., greater than 60° C.) for a period of time (e.g., greater than 10 hours) under agitation (e.g., 300 rpm). At the end of the reaction time, the solution is cooled to room temperature (e.g., by removal from the oil bath or by cooling the oil bath).

Use of an oven. In one embodiment of the present disclosure, the crystalline metal-organic framework is formed in accordance with the synthetic methods of the present disclosure by solubilizing the polytopic organic linker in a first polar protic solvent. Separately, the compound of formula MₙXₘ is dissolved in a second polar protic solvent. In some embodiments the first polar protic solvent and the second polar protic solvent are the same. In some embodiments the first polar protic solvent and the second polar protic solvent are the different. In some embodiments the polytopic organic linker solution or the MₙXₘ solution is buffered with the buffer devoid of metal coordinating functionality. In still alternative embodiments, both the polytopic organic linker solution and the MₙX^{m} solution are buffered with a buffer devoid of metal coordinating functionality. The two solutions are mixed together, for instance in a hermetically sealed autoclave with no agitation (e.g., a 200 mL sized Teflon cup autoclave) oven at room temperature. The reaction is allowed to proceed in this sealed autoclave by placing it statically into a preheated (e.g., at a temperature greater than 60° C.) for a period of time (e.g., greater than 10 hours). At the end of the reaction time, the autoclave is cooled to room temperature (e.g., by removal from the oven or by ambiently cooling the oven).

In another embodiment of the present disclosure, the crystalline metal-organic framework is formed in accordance with the synthetic methods of the present disclosure by solubilizing the polytopic organic linker in a first polar protic solvent. Separately, the compound of formula MₙXₘ is dissolved in a second polar protic solvent. In some embodiments the first polar protic solvent and the second polar protic solvent are the same. In some embodiments the first polar protic solvent and the second polar protic solvent are the different. The two solutions are mixed together, for instance in a hermetically scaled autoclave with no agitation (e.g., a 200 mL sized Teflon cup autoclave) at room temperature. The reaction is allowed to proceed in this sealed autoclave by placing it statically into a preheated (e.g., at a temperature greater than 60° C.) oven for a period of time (e.g., greater than 10 hours). At the end of the reaction time, the autoclave is cooled to room temperature along with the oven.

Silanization. In some embodiments, the disclosed reacting is performed in functionalized glassware.

Different surface functionalities of the reaction vessel can also control the resulting size and dispersity of crystals. Using literature silanizing procedures on borosilicate glassware imparts hydrophobicity to the surfaces. See Seed, 2001, “Silanizing Glassware,” Current Protocols in Cell Biology. 8:3E:A.3E.1-A.3E.2, and Plueddemann. 1992, “Chemistry of Silane Coupling Agents,” In: Silane Coupling Agents, Springer, Boston, MA, each of which is incorporated by reference. Aqueous ethanol syntheses done in these silanized glasswares have significantly lower crystal polydispersity. Further, the silanized glassware removed morphological differences stemming from different modes of heating, indicating that it can be used to mitigate synthetic inconsistencies stemming from heating variation.

In some embodiments the reacting to form the MOF crystals in accordance with the synthetic schemes of the present disclosure is performed in glassware that has been silanized with a silanizing agent. In some embodiments, the silanizing agent comprises chlorotrimethylsilane, trichlorohexylsilane, N,O-bis(trimethylsilyl)acetamide, or a mixture thereof.

In some embodiments, the silanizing agent used is (3-aminopropyl)-triethoxysilane, (3-aminopropyl)-diethoxy-methylsilane, (3-aminopropyl)-dimethyl-ethoxysilane, (3-aminopropyl)-trimethoxysilane, (3-glycidoxypropyl)-dimethyl-ethoxysilane, (3-mercaptopropyl)-trimethoxysilane, (3-mercaptopropyl)-methyl-dimethoxysilane, or a mixture thereof. In some embodiments, the silanizing agent used is one including a long hydrocarbon chain, such as octadecyltrichlorosilane, dodecyltrichlorosilane, or a mixture thereof. In some embodiments, the surface is functionalized to be more hydrophilic, rather than more hydrophobic. In some such embodiments, the silanizing agent imparts specific functionality to the surface. In some embodiments, this includes perfluoroalkanes or other alkane functionalizations such as alcohols, carboxylic acids, amides, amines, or a mixture thereof.

In some embodiments, the disclosed the reacting is performed in the presence of a benign surface.

Using benign surfaces such as plastic or steel may also lead to crystals of low dispersity. For example, aqueous ethanol syntheses done in these benign surfaces have significantly lower crystal polydispersity relative to unsilanized surfaces, such as for crystallites of Co₂(dobdc) formed in accordance with the second reaction scheme.

Further, use of different surface functionalities can determine phase selection. For example, identical reaction conditions can go to two different products depending on the surface functionality of the reaction vessel. Using different functionalities on glassware e.g. silanized or unsilanized surfaces may also lead to the discovery of new phases, such as Zn(dobpdc)·2H₂O, formed in accordance with the third reaction scheme.

Further, use of different surface functionalities can determine crystallite size. For example, identical reaction conditions can go to two different crystallite size ranges depending on the surface functionality of the reaction vessel.

### V. Technical Applications

In one aspect of the present disclosure, there is provided a number of technical applications for the disclosed adsorption materials.

One such application is carbon capture from coal flue gas or natural gas flue gas. The increasing atmospheric levels of carbon dioxide (CO₂), which are contributing to global climate change, warrant new strategies for reducing CO₂ emissions from point sources such as power plants. In particular, coal-fueled power plants are responsible for 30-40% of global CO₂ emissions. See Quadrelli et al., 2007, “The energy-climate challenge: Recent trends in CO₂ emissions from fuel combustion,” Energy Policy 35, pp. 5938-5952, which is hereby incorporated by reference. Thus, there remains a continuing need for the development of new adsorbents for carbon capture from coal flue gas, a gas stream consisting of CO₂ (15-16%), O₂ (3-4%), H₂O (5-7%), N₂ (70-75%), and trace impurities (e.g. SO₂, NOₓ) at ambient pressure and 40° C. See Planas et al, 2013, “The Mechanism of Carbon Dioxide Adsorption in an Alkylamine-Functionalized Metal-organic Framework,” J. Am. Chem. Soc. 135, pp. 7402-7405, which is hereby incorporated by reference. Similarly, growing use of natural gas as a fuel source necessitates the need for adsorbents capable of CO₂ capture from the flue gas of natural gas-fired power plants. Flue gas produced from the combustion of natural gas contains lower CO₂ concentrations of approximately 4-10% CO₂, with the remainder of the stream consisting of H₂O (saturated), O₂ (4-12%), and N₂ (balance). In particular, for a temperature swing adsorption process an adsorbent should possess the following properties: (a) a high working capacity with a minimal temperature swing, in order to minimize regeneration energy costs; (b) high selectivity for CO₂ over the other constituents of coal flue gas; (c) 90% capture of CO₂ under flue gas conditions; (d) effective performance under humid conditions; and (d) long-term stability to adsorption/desorption cycling under humid conditions.

Another such application is carbon capture from crude biogas. Biogas, the CO₂/CH₄ mixtures produced by the breakdown of organic matter, is a renewable fuel source with the potential to replace traditional fossil fuel sources. Removal of CO₂ from the crude biogas mixtures is one of the most challenging aspects of upgrading this promising fuel source to pipeline quality methane. Therefore, the use of adsorbents to selectively remove CO₂ from CO₂/CH₄ mixtures with a high working capacity and minimal regeneration energy has the potential to greatly reduce the cost of using biogas in place of natural gas for applications in the energy sector.

The disclosed compositions (adsorption materials) can be used to strip a major portion of the CO₂ from the CO₂-rich gas stream, and the adsorption material enriched for CO₂ can be stripped of CO₂ using a temperature swing adsorption method, a pressure swing adsorption method, a vacuum swing adsorption method, a concentration swing adsorption method, or a combination thereof. Example temperature swing adsorption methods and vacuum swing adsorption methods are disclosed in International Publication Number WO2013/059527 A1, which is hereby incorporated by reference.

In some embodiments, the disclosed compositions (adsorption materials) are used to separate hydrocarbon mixtures such as ethane/ethylene, propane/propylene, and C₆ alkane mixtures, among many others. Industrial production of these hydrocarbons produces mixtures of the olefin/paraffin types or other isomers, which do not match market demand and must be separated. Some of the current technologies are very energy-intensive processes such as distillation, and some are crystallization or adsorption-based. Implementing better adsorption-based materials has the potential to greatly reduce energy costs in industrial separations.

In some embodiments, the disclosed compositions are used as heterogeneous catalysts for the conversion of light alkanes into value-added chemicals, among other processes, including the conversion of methane. Given the recent worldwide increase in natural gas reserves, this process is one with tremendous economic and environmental impacts. Therefore, materials and routes for converting methane to higher hydrocarbons are highly desired.

### VI. Examples

Example 1. A crystalline metal-organic framework comprising a plurality of cations and a plurality of polytopic organic linkers was synthesized, where each polytopic organic linker in the plurality of polytopic organic linkers was connected to two or more cations in the plurality of cations. The polytopic organic linkers had the formula:

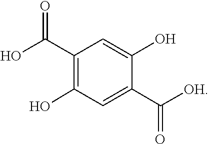 [SMILES: O=C(O)c1cc(O)c(C(=O)O)cc1O]

The polytopic organic linkers were reacted with a compounds of formula Co(NO₃)₂·6H₂O in a 1:1 H₂O/EtOH solution buffered with 0.2 mM MOPS adjusted to pH 7. The concentration of the polytopic organic linkers in the solution was 5 mM and the concentration of Co(NO₃)₂·6H₂O in the solution was 17.5 mM. Present in the solution was a modulator of formula:

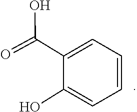

The concentration of the modulator in the solution was 35 mM. The solution was held at a temperature of 75° C. during the reaction thereby affording CO₂(dobdc) crystals illustrated in the lower panel of FIG. 8A, top panel.

Example 2. A crystalline metal-organic framework comprising a plurality of cations and a plurality of polytopic organic linkers was synthesized, where each polytopic organic linker in the plurality of polytopic organic linkers was connected to two or more cations in the plurality of cations. The polytopic organic linkers had the formula:

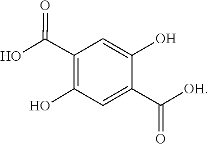 [SMILES: O=C(O)c1cc(O)c(C(=O)O)cc1O]

The polytopic organic linkers were reacted with a compounds of formula Co(NO₃)₂·6H₂O in a 1:1 H₂O/EtOH solution buffered with 0.2 mM MOPS adjusted to pH 7. The concentration of the polytopic organic linkers in the solution was 5 mM and the concentration of Co(NO₃)₂·6H₂O in the solution was 17.5 mM. Present in the solution was a modulator of formula:

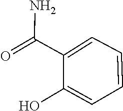

The concentration of the modulator in the solution was 35 mM. The solution was held at a temperature of 75° C. during the reaction thereby affording Co₂(dobdc) crystals illustrated in the lower panel of FIG. 8A, bottom panel.

Example 3. A crystalline metal-organic framework comprising a plurality of cations and a plurality of polytopic organic linkers was synthesized, where each polytopic organic linker in the plurality of polytopic organic linkers was connected to two or more cations in the plurality of cations. The polytopic organic linkers had the formula:

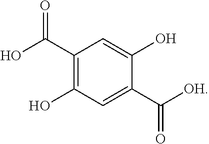 [SMILES: O=C(O)c1cc(O)c(C(=O)O)cc1O]

The polytopic organic linkers were reacted with a compounds of formula Co(NO₃)₂·6H₂O in a 1:1 H₂O/EtOH solution buffered with 0.2 mM MOPS adjusted to pH 7. The concentration of the polytopic organic linkers in the solution was 5 mM and the concentration of Co(NO₃)₂·6H₂O in the solution was 17.5 mM. Present in the solution was a modulator of formula:

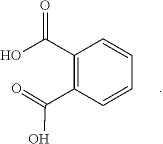 [SMILES: O=C(O)c1ccccc1C(=O)O]

The concentration of the modulator in the solution was 35 mM. The solution was held at a temperature of 75° C. during the reaction thereby affording Co₂(dobdc) crystals illustrated in the lower panel of FIG. 8B, top panel.

Example 4. A crystalline metal-organic framework comprising a plurality of cations and a plurality of polytopic organic linkers was synthesized, where each polytopic organic linker in the plurality of polytopic organic linkers was connected to two or more cations in the plurality of cations. The polytopic organic linkers had the formula:

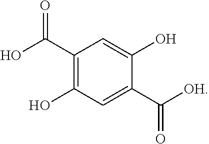 [SMILES: O=C(O)c1cc(O)c(C(=O)O)cc1O]

The polytopic organic linkers were reacted with a compounds of formula Co(NO₃)₂·6H₂O in a 1:1 H₂O/EtOH solution buffered with 0.2 mM MOPS adjusted to pH 7. The concentration of the polytopic organic linkers in the solution was 5 mM and the concentration of Co(NO₃)₂·6H₂O in the solution was 17.5 mM. Present in the solution was a modulator of formula:

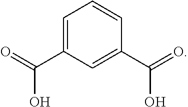 [SMILES: O=C(O)c1cccc(C(=O)O)c1]

The concentration of the modulator in the solution was 35 mM. The solution was held at a temperature of 75° C. during the reaction thereby affording Co₂(dobdc) crystals illustrated in the lower panel of FIG. 8B, bottom panel.

Example 5. A crystalline metal-organic framework comprising a plurality of cations and a plurality of polytopic organic linkers was synthesized, where each polytopic organic linker in the plurality of polytopic organic linkers was connected to two or more cations in the plurality of cations. The polytopic organic linkers had the formula:

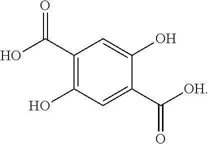 [SMILES: O=C(O)c1cc(O)c(C(=O)O)cc1O]

The polytopic organic linkers were reacted with a compounds of formula Co(NO₃)₂·6H₂O in a 1:1 H₂O/EtOH solution buffered with 0.2 mM MOPS adjusted to pH 7. The concentration of the polytopic organic linkers in the solution was 5 mM and the concentration of Co(NO₃)₂·6H₂O in the solution was 17.5 mM. Present in the solution was a modulator of formula:

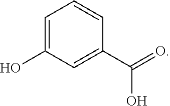

The concentration of the modulator in the solution was 35 mM. The solution was held at a temperature of 75° C. during the reaction thereby affording Co₂(dobdc) crystals illustrated in the lower panel of FIG. 8C.

### VII. Additional Embodiments

Embodiment 1. A method of synthesizing a crystalline metal-organic framework comprising a plurality of cations and a plurality of polytopic organic linkers, wherein each polytopic organic linker in the plurality of polytopic organic linkers is connected to two or more cations in the plurality of cations, the method comprising: reacting the plurality of polytopic organic linkers with one or more compounds of formula MₙXₘ in a solution, wherein each M is independently cationic Be, Mg, Ca, Ti, V, Cr, Mn, Fe, Co, Ni, Cu, Zn, Zr, Nb, Mo, Ru, Rh, Pd, Cd, Hf, X is a basic anion, n is a positive integer, m is a positive integer, and the reacting is in the presence of a modulator having the formula:

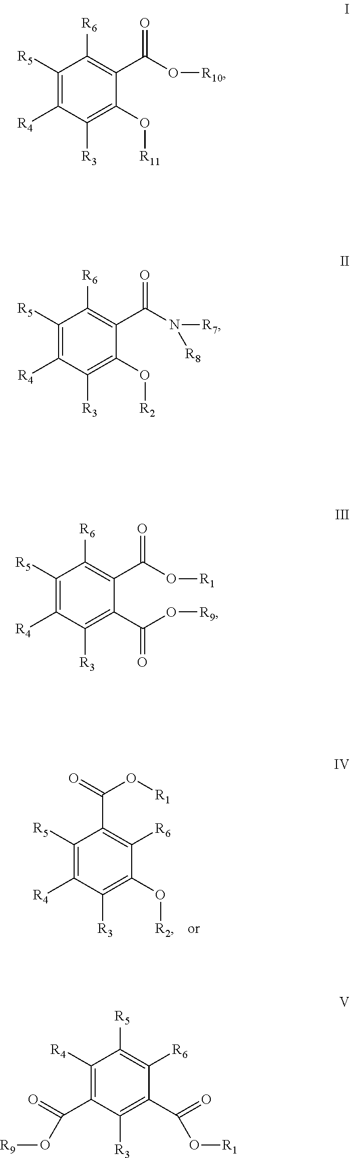

or a salt (e.g., sodium, potassium cesium) thereof or a mixture thereof wherein R₁, R₂, R₇, R₈, R₉, R₁₀ and R₁₁ are each independently selected from hydrogen, halogen, substituted or unsubstituted alkyl, substituted or unsubstituted heteroalkyl, substituted or unsubstituted aryl, substituted or unsubstituted heteroaryl, or substituted or unsubstituted heterocycloalkyl, R₃·R₄, R₅, and R₆ are each independently selected from H, halogen, hydroxyl, methyl, or halogen substituted methyl, and wherein R₁₀ and R₁₁ are not each hydrogen.

Embodiment 2. The method of embodiment 1, wherein R₃, R₄, R₅, and R₆ are each independently a substituted or unsubstituted linear or branched alkyl having between one and ten carbon atoms.

Embodiment 3. The method of embodiment 1, wherein R₃, R₄, R₅, and R₆ are each hydrogen.

Embodiment 4. The method of embodiment 1 or 3, wherein the modulator has formula IV and R₁ and R₂ are each hydrogen.

Embodiment 5. The method of embodiment 1 or 3, wherein the modulator has formula II and R₂, R₇ and R₈ are each hydrogen.

Embodiment 6. The method of embodiment 1 or 3, wherein the modulator has formula III or V and R₁ and R₉ are each hydrogen.

Embodiment 7. The method of any one of embodiments 1-6, wherein the reacting is performed with a buffer devoid of metal coordinating functionality.

Embodiment 8. The method of embodiment 7, wherein the buffer devoid of metal coordinating functionality is PIPES, PIPPS, PIPBS, DEPP, DESPEN, MES, TEEN, PIPES, MOBS, DESPEN, or TEMN.

Embodiment 9. The method of embodiment 7, wherein the buffer devoid of metal coordinating functionality is an alkyl or alkylsulfonate derivative of morpholine, piperazine, ethylenediamine, or methylenediamine.

Embodiment 10. The method of any one of embodiments 1-9, wherein each polytopic organic linker in the plurality of polytopic organic linkers has the formula:

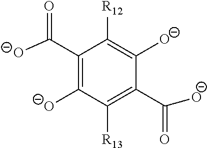

wherein, R₁₂ and R₁₃ are each independently selected from H, halogen, hydroxyl, methyl, or halogen substituted methyl.

Embodiment 11. The method of embodiment 10, wherein R₁₂ and R₁₃ are each hydrogen.

Embodiment 12. The method of any one of embodiments 1-9, wherein each polytopic organic linker in the plurality of polytopic organic linkers has the formula:

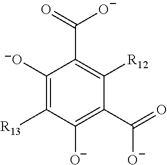

wherein R₁₂ and R₁₃ are each independently selected from H, halogen, hydroxyl, methyl, or halogen substituted methyl.

Embodiment 13. The method of embodiment 12, wherein R₁₂ and R₁₃ are each hydrogen.

Embodiment 14. The method of any one of embodiments 1-9, wherein each polytopic organic linker in the plurality of polytopic organic linkers has the formula:

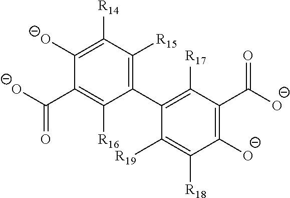

wherein R₁₄, R₁₅, R₁₆, R₁₇, R₁₈, and R₁₉ are each independently selected from H, halogen, hydroxyl, methyl, or halogen substituted methyl.

Embodiment 15. The method of embodiment 14, wherein R₁₄, R₁₅, R₁₆, R₁₇, R₁₈, and R₁₉ are each hydrogen.

Embodiment 16. The method of any one of embodiments 1-9, wherein each polytopic organic linker in the plurality of polytopic organic linkers has the formula:

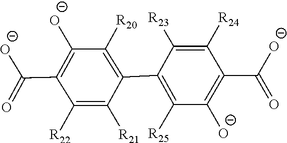

wherein R₂₀, R₂₁, R₂₂, R₂₃, R₂₄, and R₂₅ are each independently selected from H, halogen, hydroxyl, methyl, or halogen substituted methyl.

Embodiment 17. The method of embodiment 16, wherein R₂₀, R₂₁, R₂₂, R₂₃, R₂₄, and R₂₅ are each hydrogen.

Embodiment 18. The method of any one of embodiments 1-9, wherein each polytopic organic linker in the plurality of polytopic organic linkers is: 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), dioxidobiphenyl-4,4′-dicarboxylate (para-carboxylate-dobpdc⁴⁻), also referred to as pc-dobpdc⁴⁻), 2,5-dioxidobenzene-1,4-dicarboxylate (dobdc⁴⁻), 4,6-dioxido-1,3-benzenedicarboxylate (m-dobdc⁴⁻), 1,3,5-benzenetristetrazolate (BTT), 1,3,5-benzenetristriazolate (BTTri), 1,3,5-benzenetrispyrazolate (BTP), or 1,3,5-benzenetriscarboxylate (BTC).

Embodiment 19. The method of any one of embodiments 1-9, wherein each polytopic organic linker in the plurality of polytopic organic linkers is 2,5-dioxido-1,4-benzenedicarboxylate (dobdc⁴⁻).

Embodiment 20. The method of any one of embodiments 1-19, wherein a compound in the one or more compounds of formula MₙXₘ is a magnesium(II) metal salt, a manganese(II) metal salt, an iron(11) metal salt, a cobalt (II) metal salt, a nickle(II) metal salt, a zinc(II) metal salt, or a cadmium(II) metal salt.

Embodiment 21. The method of any one of embodiments 1-19, wherein a compound in the one or more compounds of formula MₙXₘ is cobalt(II) nitrate, cobalt(II) chloride, cobalt(II) acetate, cobalt(II) sulfate, cobalt(II) iodide, cobalt(II) bromide, cobalt(II) trifluorosulfonate, cobalt(II) tetrafluoroborate, cobalt(II) oxide, cobalt(II) carbonate, cobalt(II) hydroxide, cobalt(II) hydroxycarbonate, mixed-halide cobalt(II), cobalt(II) acetylacetonate, cobalt(II) formate, or a halogenated derivative thereof.

Embodiment 22. The method of embodiment 1, wherein the pKa of the anion is above the lowest pKa value of the polytopic organic linker.

Embodiment 23. The method of any one of embodiments 1-22, wherein the basic anion is formate, acetate, sulfate, bromide, iodide, or triflurosulfonate.

Embodiment 24. The method of any one of embodiments 1-23, wherein the reacting is performed in unsilanized glassware using an oil bath.

Embodiment 25. The method of any one of embodiments 1-23, wherein the reacting is performed in functionalized glassware.

Embodiment 26. The method of any one of embodiments 1-23, wherein the reacting is performed in the presence of a benign surface.

Embodiment 27. The method of any one of embodiments 1-23, wherein the reacting is performed in glassware that has been silanized with a silanizing agent.

Embodiment 28. The method of embodiment 27, wherein the silanizing agent comprises chlorotrimethylsilane, trichlorohexylsilane, N,O-bis(trimethylsilyl)acetamide, or a mixture thereof.

Embodiment 29. The method of any one of embodiments 1-28, wherein the reacting occurs in a 1:1 ethanol:H₂O solvent.

Embodiment 30. The method of any one of embodiments 1-29, wherein the reacting occurs at a temperature greater than 25° C. for at least one hour.

Embodiment 31. The method of any one of embodiments 1-29, wherein the reacting is performed at a temperature greater than 25° C. for at least eight hours.

Embodiment 32. The method of any one of embodiments 1-31, wherein n is 1 and m either 1 or 2.

Embodiment 33. The method of any one of embodiments 1-31, wherein m is 2 or greater.

Embodiment 34. The method of any one of embodiments 1-33, wherein each polytopic organic linker in the plurality of polytopic organic linkers is connected to two metal cations in the plurality of metal cations.

Embodiment 35. The method of any one of embodiments 1-34, wherein there is one equivalent of the polytopic organic linker to between 0.5 and 20 equivalents of the modulator in the solution prior to the reacting.

Embodiment 36. The method of any one of embodiments 1-34, wherein there is one equivalent of the polytopic organic linker to between 1 and 15 equivalents of the modulator in the solution prior to the reacting.

Embodiment 37. The method of any one of embodiments 1-36, wherein a pH of the solution is between 6.5 and 8.5.

Embodiment 38. The method of any one of embodiments 1-36, wherein a pH of the solution is between 7.0 and 8.0.

Embodiment 39. The method of embodiment 1, wherein the solution comprises t-butanol, n-propanol, ethanol, methanol, acetic acid, water, N,N-dimethylformamide or a mixture thereof.

Embodiment 40. The method of any one of embodiments 1-39, wherein the plurality of polytopic organic linker is present in the solution at a concentration of between 1 mM and 50 mM prior to the reacting, the one or more compounds of formula MₙXₘ is present in the solution at a concentration of between 5 nM and 100 nM prior to the reacting, and the modulator is present in the solution at a concentration of between 15 nM and 100 nM prior to the reacting.

Embodiment 41. The method of any one of embodiments 1-40, wherein the reacting is performed at a temperature greater than 60° C.

Embodiment 42. The method of any one of embodiments 1-40, wherein the reacting is performed at a temperature between 70° C. and 80° C.

Embodiment 43. The method of any one of embodiments 1-42, wherein the salt of the modulator is a sodium salt, a potassium salt, or a cesium salt.

### CONCLUSION

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of synthesizing a crystalline metal-organic framework comprising a plurality of cations and a plurality of polytopic organic linkers, wherein each polytopic organic linker in the plurality of polytopic organic linkers is connected to two or more cations in the plurality of cations, the method comprising:
reacting the plurality of polytopic organic linkers with one or more compounds of formula MₙXₘ in a solution, wherein
each M is independently cationic Be, Mg, Ca, Ti, V, Cr, Mn, Fe, Co, Ni, Cu, Zn, Zr, Nb, Mo, Ru, Rh, Pd, Cd, or Hf,
X is a basic anion,
n is a positive integer,
m is a positive integer, and
the reacting is in the presence of a modulator having the formula: 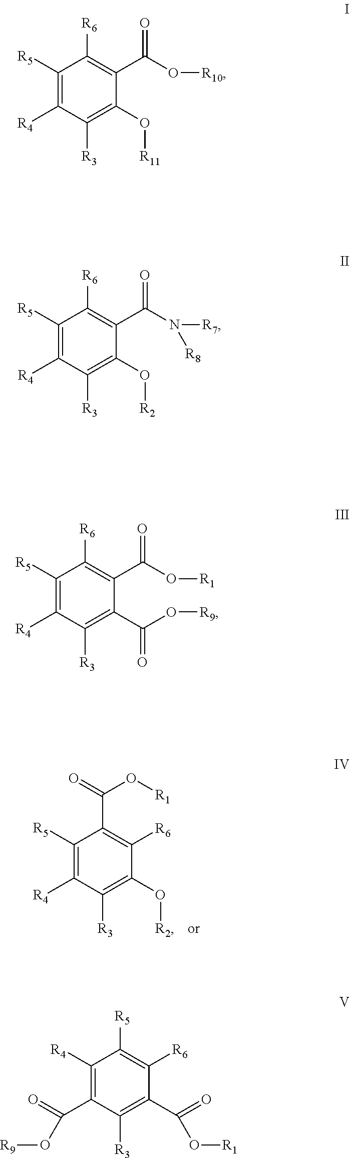
or a salt thereof, or a mixture thereof wherein,
R₁, R₂, R₇, R₈, R₉, R₁₀ and R₁₁ are each independently selected from hydrogen, halogen, substituted or unsubstituted alkyl, substituted or unsubstituted heteroalkyl, substituted or unsubstituted aryl, substituted or unsubstituted heteroaryl, or substituted or unsubstituted heterocycloalkyl,
R₃, R₄, R₅, and R₆ are each independently selected from H, halogen, hydroxyl, methyl, or halogen substituted methyl, and
wherein R₁₀ and R₁₁ are not each hydrogen.

2. The method of claim 1, wherein R₃, R₄, R₅, and R₆ are each independently a substituted or unsubstituted linear or branched alkyl having between one and ten carbon atoms.

3. The method of claim 1, wherein R₃, R₄, R₅, and R₆ are each hydrogen.

4. The method of claim 1, wherein the modulator has formula IV and R₁ and R₂ are each hydrogen.

5. The method of claim 1, wherein the modulator has formula II and R₂, R₇ and R₈ are each hydrogen.

6. The method of claim 1, wherein the modulator has formula III or V and R₁ and R₉ are each hydrogen.

7. The method of claim 1, wherein each polytopic organic linker in the plurality of polytopic organic linkers has the formula: 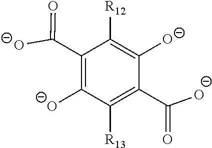
wherein, R₁₂ and R₁₃ are each independently selected from H, halogen, hydroxyl, methyl, or halogen substituted methyl.

8. The method of claim 1, wherein each polytopic organic linker in the plurality of polytopic organic linkers has the formula: 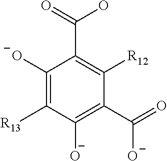
wherein R₁₂ and R₁₃ are each independently selected from H, halogen, hydroxyl, methyl, or halogen substituted methyl.

9. The method of claim 1, wherein each polytopic organic linker in the plurality of polytopic organic linkers has the formula: 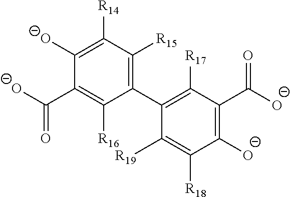
wherein R₁₄, R₁₅, R₁₆, R₁₇, R₁₈, and R₁₉ are each independently selected from H, halogen, hydroxyl, methyl, or halogen substituted methyl.

10. The method of claim 1, wherein each polytopic organic linker in the plurality of polytopic organic linkers has the formula: 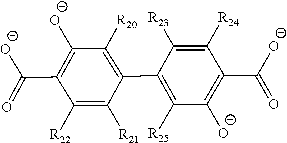
wherein R₂₀, R₂₁, R₂₂, R₂₃, R₂₄, and R₂₅ are each independently selected from H, halogen, hydroxyl, methyl, or halogen substituted methyl.

11. The method of claim 1, wherein each polytopic organic linker in the plurality of polytopic organic linkers is 2,5-dioxido-1,4-benzenedicarboxylate (dobdc⁴⁻).

12. The method of claim 1, wherein a compound in the one or more compounds of formula MₙXₘ is a magnesium(II) metal salt, a manganese(II) metal salt, an iron(II) metal salt, a cobalt (II) metal salt, a nickle(II) metal salt, a zinc(II) metal salt, a cadmium(II) metal salt.

13. The method of claim 1, wherein a compound in the one or more compounds of formula MₙXₘ is cobalt(II) nitrate, cobalt(II) chloride, cobalt(II) acetate, cobalt(II) sulfate, cobalt(II) iodide, cobalt(II) bromide, cobalt(II) trifluorosulfonate, cobalt(II) tetrafluoroborate, cobalt(II) oxide, cobalt(II) carbonate, cobalt(II) hydroxide, cobalt(II) hydroxycarbonate, mixed-halide cobalt(II), cobalt(II) acetylacetonate, cobalt(II) formate, or a halogenated derivative thereof.

14. The method of claim 1, wherein the basic anion is formate, acetate, sulfate, bromide, iodide, or triflurosulfonate.

15. The method of claim 1, wherein n is 1 and m either 1 or 2 and a pH of the solution is between 7.0 and 8.0.

16. The method of claim 1, wherein m is 2 or greater and a pH of the solution is between 7.0 and 8.0.

17. The method of claim 1, wherein each polytopic organic linker in the plurality of polytopic organic linkers is connected to two metal cations in the plurality of metal cations.

18. The method of claim 1, wherein there is one equivalent of the polytopic organic linker to between 0.5 and 20 equivalents of the modulator in the solution prior to the reacting.

19. The method of claim 1, wherein there is one equivalent of the polytopic organic linker to between 1 and 15 equivalents of the modulator in the solution prior to the reacting.

20. The method of claim 1, wherein
the plurality of polytopic organic linkers is present in the solution at a concentration of between 1 mM and 50 mM prior to the reacting,
the one or more compounds of formula MₙXₘ is present in the solution at a concentration of between 5 nM and 100 nM prior to the reacting, and
the modulator is present in the solution at a concentration of between 15 nM and 100 nM prior to the reacting.
